SEQUENCE LISTING (128 sequences)

SEQ ID NO: 1 - DNA, 18 bp, Artificial Sequence
  telomerase specific antisense primer
aatccgtcgagcagagtt

SEQ ID NO: 2 - DNA, 20 bp, Artificial Sequence
  Rex1 sense primer
tgaaagcccacatcctaacg

SEQ ID NO: 3 - DNA, 20 bp, Artificial Sequence
  Rex1 antisense primer
caagctatcctcctgctttg

SEQ ID NO: 4 - DNA, 19 bp, Artificial Sequence
  Oct4 sense primer
acatgtgtaagctgcggcc

SEQ ID NO: 5 - DNA, 21 bp, Artificial Sequence
  Oct4 antisense primer
gttgtgcatagtcgctgcttg

SEQ ID NO: 6 - DNA, 22 bp, Artificial Sequence
  Oct4 sense primer
ctacgggacatcctctggctcc

SEQ ID NO: 7 - DNA, 22 bp, Artificial Sequence
  Oct4 antisense primer
catctctgccagcagcgtgctg

SEQ ID NO: 8 - DNA, 21 bp, Artificial Sequence
  CDK1 sense primer
ggctcttggaaattgagcgga

SEQ ID NO: 9 - DNA, 21 bp, Artificial Sequence
  CDK1 antisense primer
aggaaccccttcctcttcact

SEQ ID NO: 10 - DNA, 22 bp, Artificial Sequence
  CDK2 sense primer
ctagctttctgccattctcatc

SEQ ID NO: 11 - DNA, 22 bp, Artificial Sequence
  CDK2 antisense primer
gaagagctggtcaatctcagaa

SEQ ID NO: 12 - DNA, 24 bp, Artificial Sequence
  beta actin sense primer
tttgagaccttcaacaccccagcc

SEQ ID NO: 13 - DNA, 22 bp, Artificial Sequence
  beta actin antisense primer
aatgtcacgcacgatttcccgc

SEQ ID NO: 14 - DNA, 25 bp, Artificial Sequence
  Nanog sense primer
tctgtttcttgactgggaccttgtc

SEQ ID NO: 15 - DNA, 21 bp, Artificial Sequence
  Nanog antisense primer
gctgagatgcctcacacggag

SEQ ID NO: 16 - DNA, 24 bp, Artificial Sequence
  Utf1 sense primer
ccgtcgctgaacaacgccctgctg

SEQ ID NO: 17 - DNA, 24 bp, Artificial Sequence
  Utf1 antisense primer
cgcgctgcccagaatgaagcccac

SEQ ID NO: 18 - DNA, 20 bp, Artificial Sequence
  Dapp5 sense primer
tgaaagatccagaggtgttc

SEQ ID NO: 19 - DNA, 20 bp, Artificial Sequence
  Dapp5 antisense primer
actggttcacttcatccaag

SEQ ID NO: 20 - DNA, 20 bp, Artificial Sequence
  FGF4 sense primer
ctacaacgcctacgagtcct

SEQ ID NO: 21 - DNA, 20 bp, Artificial Sequence
  FGF4 antisense primer
gttgcaccagaaaagtcaga

SEQ ID NO: 22 - DNA, 20 bp, Artificial Sequence
  ERas sense primer
gctgtctgtgatggtgtgct

SEQ ID NO: 23 - DNA, 20 bp, Artificial Sequence
  ERas antisense primer
tctccagcagtggtcacaag

SEQ ID NO: 24 - DNA, 24 bp, Artificial Sequence
  M13 antisense primer
agcggataacaatttcacacagga

SEQ ID NO: 25 - DNA, 23 bp, Artificial Sequence
  OCT4 1 sense primer
tttttagttttttttaggtttaa

SEQ ID NO: 26 - DNA, 20 bp, Artificial Sequence
  OCT4 1 antisense primer
taaacaaaaaacccattccc

SEQ ID NO: 27 - DNA, 25 bp, Artificial Sequence
  OCT4 2 sense primer
ttaggaaaatgggtagtagggattt

SEQ ID NO: 28 - DNA, 30 bp, Artificial Sequence
  OCT4 2 antisense primer
tacccaaaaaacaaataaattataaaacct

SEQ ID NO: 29 - DNA, 25 bp, Artificial Sequence
  OCT4 3 sense primer
atttgttttttgggtagttaaaggt

SEQ ID NO: 30 - DNA, 27 bp, Artificial Sequence
  OCT4 3 antisense primer
ccaactatcttcatcttaataacatcc

SEQ ID NO: 31 - DNA, 27 bp, Artificial Sequence
  OCT4 4 sense primer
ggatgttattaagatgaagatagttgg

SEQ ID NO: 32 - DNA, 25 bp, Artificial Sequence
  OCT4 4 antisense primer
cctaaactccccttcaaaatctatt

SEQ ID NO: 33 - DNA, 24 bp, Artificial Sequence
  OCT4 5 sense primer
gaaggggaagtagggattaatttt

SEQ ID NO: 34 - DNA, 25 bp, Artificial Sequence
  OCT4 5 antisense primer
caacaaccataaacacaataaccaa

SEQ ID NO: 35 - DNA, 25 bp, Artificial Sequence
  OCT4 6 sense primer
tagttgggatgtgtagagtttgaga

SEQ ID NO: 36 - DNA, 25 bp, Artificial Sequence
  OCT4 6 antisense primer
taaaccaaaacaatccttctactcc

SEQ ID NO: 37 - DNA, 25 bp, Artificial Sequence
  OCT4 7 sense primer 1
ataaagtgagattttgttttaaaaa

SEQ ID NO: 38 - DNA, 22 bp, Artificial Sequence
  OCT4 7 antisense primer 1
aacataaaaaaatcccccacac

SEQ ID NO: 39 - DNA, 22 bp, Artificial Sequence
  OCT4 7 sense primer 2
gggatttgtattgaggttttgg

SEQ ID NO: 40 - DNA, 20 bp, Artificial Sequence
  OCT4 7 antisense primer 2
cccacacctcaaaacctaac

SEQ ID NO: 41 - DNA, 25 bp, Artificial Sequence
  NANOG 1 sense primer
agagataggagggtaagtttttttt

SEQ ID NO: 42 - DNA, 26 bp, Artificial Sequence
  NANOG 1 antisense primer
actcccacacaaactaacttttattc

SEQ ID NO: 43 - DNA, 30 bp, Artificial Sequence
  NANOG 2 sense primer
gagttaaagagttttgtttttaaaaattat

SEQ ID NO: 44 - DNA, 30 bp, Artificial Sequence
  NANOG 2 antisense primer
tcccaaatctaataatttatcatatctttc

SEQ ID NO: 45 - DNA, 25 bp, Artificial Sequence
  NANOG 3sense primer
ttaatttattgggattataggggtg

SEQ ID NO: 46 - DNA, 24 bp, Artificial Sequence
  NANOG 3 antisense primer
aacaacaaaacctaaaaacaaacc

SEQ ID NO: 47 - DNA, 23 bp, Artificial Sequence
  SOX2 1sense primer
gtaggttggttttgggagttttt

SEQ ID NO: 48 - DNA, 26 bp, Artificial Sequence
  SOX2 1 antisense primer
aattaataaacaaccatccatataac

SEQ ID NO: 49 - DNA, 28 bp, Artificial Sequence
  SOX2 2 sense primer
tgtttttttaagattaggattgagagaa

SEQ ID NO: 50 - DNA, 25 bp, Artificial Sequence
  SOX2 2 antisense primer
aaaacaaactaaaatcaaaatcaaa

SEQ ID NO: 51 - DNA, 20 bp, Artificial Sequence
  SOX2 3 sense primer
acaaactaactctaaaaacc

SEQ ID NO: 52 - DNA, 20 bp, Artificial Sequence
  SOX2 3 antisense primer
ggttgttagggaataaatgg

SEQ ID NO: 53 - DNA, 30 bp, Artificial Sequence
  SOX2 4 sense primer
agatggtttaggagaattttaagatgtata

SEQ ID NO: 54 - DNA, 30 bp, Artificial Sequence
  SOX2 4 antisense primer
aacccaactaatcctacatcatactataac

SEQ ID NO: 55 - DNA, 30 bp, Artificial Sequence
  SOX2 5 sense primer
ggtagttatagtatgatgtaggattagttg

SEQ ID NO: 56 - DNA, 23 bp, Artificial Sequence
  SOX2 5 antisense primer
aacccataaaaccaaaaaccata

SEQ ID NO: 57 - DNA, 25 bp, Artificial Sequence
  SOX2 6 sense primer
gggatatgattagtatgtatttttt

SEQ ID NO: 58 - DNA, 30 bp, Artificial Sequence
  SOX2 6 antisense primer
aattttctccatactatttcttactctcct

SEQ ID NO: 59 - DNA, 25 bp, Artificial Sequence
  REX1 1 sense primer
aaatattgggggtgtttgaaataat

SEQ ID NO: 60 - DNA, 26 bp, Artificial Sequence
  REX1 1 antisense primer
cccaactactcaaaaaactaaaacaa

SEQ ID NO: 61 - DNA, 25 bp, Artificial Sequence
  REX1 2 sense primer
aaaagggtaaatgtgattatattta

SEQ ID NO: 62 - DNA, 22 bp, Artificial Sequence
  REX1 2 antisense primer
caaactacaaccacccatcaac

SEQ ID NO: 63 - DNA, 25 bp, Artificial Sequence
  REX1 3 sense primer
atgggtggttgtagtttgattagat

SEQ ID NO: 64 - DNA, 26 bp, Artificial Sequence
  REX1 3 antisense primer
tttcaacatttaaaaccaataaccaa

SEQ ID NO: 65 - DNA, 30 bp, Artificial Sequence
  REX1 4 sense primer
ttattataaaagagttaggaagtttgtata

SEQ ID NO: 66 - DNA, 25 bp, Artificial Sequence
  REX1 4 antisense primer
attacccaaactaaaatacaacaac

SEQ ID NO: 67 - DNA, 25 bp, Artificial Sequence
  REX1 5 sense primer
tttggaggaatatttggtattgatt

SEQ ID NO: 68 - DNA, 27 bp, Artificial Sequence
  REX1 5 antisense primer
cctattacaaccttaaaaaaaacacac

SEQ ID NO: 69 - DNA, 19 bp, Artificial Sequence
  TERT sense primer
ctaccccttcaccttccaa

SEQ ID NO: 70 - DNA, 20 bp, Artificial Sequence
  TERT antisense primer
gttagttttggggttttagg

SEQ ID NO: 71 - DNA, 25 bp, Artificial Sequence
  VEGFA 1 sense primer
gttattatagggaagttgggtgaat

SEQ ID NO: 72 - DNA, 25 bp, Artificial Sequence
  VEGFA 1 antisense primer
ccaaaattcacaacctaaaaattac

SEQ ID NO: 73 - DNA, 22 bp, Artificial Sequence
  VEGFA 2 sense primer
taggttgtgaattttggtgggg

SEQ ID NO: 74 - DNA, 23 bp, Artificial Sequence
  VEGFA 2 antisense primer
aaataaaacaatctccccaaacc

SEQ ID NO: 75 - DNA, 26 bp, Artificial Sequence
  VEGFA 3 sense primer
aggtagtaagagttttagagagaagt

SEQ ID NO: 76 - DNA, 21 bp, Artificial Sequence
  VEGFA 3 antisense primer
aaaacaacccaaaaattaaac

SEQ ID NO: 77 - DNA, 26 bp, Artificial Sequence
  VEGFA 4 sense primer
tgggtgtattggagttttgttttgtt

SEQ ID NO: 78 - DNA, 25 bp, Artificial Sequence
  VEGFA 4 antisense primer
ccctacccactaatctctaactccc

SEQ ID NO: 79 - DNA, 25 bp, Artificial Sequence
  NESTIN 1 sense primer
atttaatattttttggtagggggtg

SEQ ID NO: 80 - DNA, 30 bp, Artificial Sequence
  NESTIN 1 antisense primer
cctaaaaaacaaaaacaaatatctaatatt

SEQ ID NO: 81 - DNA, 25 bp, Artificial Sequence
  NESTIN 2 sense primer
gtttgtttatttttagtgggttaga

SEQ ID NO: 82 - DNA, 24 bp, Artificial Sequence
  NESTIN 2 antisense primer
ctccaactcttcaaccaaattatc

SEQ ID NO: 83 - DNA, 22 bp, Artificial Sequence
  RUNX3 1 sense primer
attttggaggatttgttttggg

SEQ ID NO: 84 - DNA, 20 bp, Artificial Sequence
  RUNX3 1 antisense primer
caacctacccractaatccc

SEQ ID NO: 85 - DNA, 22 bp, Artificial Sequence
  RUNX3 2 sense primer
ttaygaggggyggtygtaygyg

SEQ ID NO: 86 - DNA, 24 bp, Artificial Sequence
  RUNX3 2 antisense primer
aaaacraccracrcraacrcctcc

SEQ ID NO: 87 - DNA, 23 bp, Artificial Sequence
  CDK2 1 sense primer
ttaaagtaggtatttgggaagag

SEQ ID NO: 88 - DNA, 26 bp, Artificial Sequence
  CDK2 1 antisense primer
tttaaccaacttaaaacaatattacc

SEQ ID NO: 89 - DNA, 25 bp, Artificial Sequence
  CDK2 2 sense primer
tttaagttggttaaattgataagag

SEQ ID NO: 90 - DNA, 26 bp, Artificial Sequence
  CDK2 2 antisense primer
attaaaaaaaacaatcaaaaaaatcc

SEQ ID NO: 91 - DNA, 29 bp, Artificial Sequence
  TP53 sense primer
aaaaattgaagtttatagaggttaagggt

SEQ ID NO: 92 - DNA, 25 bp, Artificial Sequence
  TP53 antisense primer
cacaaacccaaaataaaaccaatac

SEQ ID NO: 93 - DNA, 24 bp, Artificial Sequence
  Utf1 sense primer
ccgtcgctgaacaacgccctgctg

SEQ ID NO: 94 - DNA, 24 bp, Artificial Sequence
  Utf1 antisense primer
cgcgctgcccagaatgaagcccac

SEQ ID NO: 95 - DNA, 20 bp, Artificial Sequence
  Dapp5 sense primer
tgaaagatccagaggtgttc

SEQ ID NO: 96 - DNA, 20 bp, Artificial Sequence
  Dapp5 antisense primer
actggttcacttcatccaag

SEQ ID NO: 97 - DNA, 20 bp, Artificial Sequence
  FGF4 sense primer
ctacaacgcctacgagtcct

SEQ ID NO: 98 - DNA, 20 bp, Artificial Sequence
  FGF4 antisense primer
gttgcaccagaaaagtcaga

SEQ ID NO: 99 - DNA, 20 bp, Artificial Sequence
  ERas sense primer
gctgtctgtgatggtgtgct

SEQ ID NO: 100 - DNA, 20 bp, Artificial Sequence
  ERas antisense primer
tctccagcagtggtcacaag

SEQ ID NO: 101 - DNA, 25 bp, Artificial Sequence
  Cyclin2 sense primer
gagaagctgtccctgatccgcaagc

SEQ ID NO: 102 - DNA, 24 bp, Artificial Sequence
  Cyclin2 antisense primer
agacttggagccgttgtgctgctc

SEQ ID NO: 103 - DNA, 20 bp, Artificial Sequence
  MMP2 sense primer
acgaccgcgacaagaactat

SEQ ID NO: 104 - DNA, 22 bp, Artificial Sequence
  MMP2 antisense primer
ctgcaaagaacacagccttctc

SEQ ID NO: 105 - DNA, 18 bp, Artificial Sequence
  MMP9 sense primer
cccgtcctgctttgcagt

SEQ ID NO: 106 - DNA, 25 bp, Artificial Sequence
  MMP9 antisense primer
atccaagtttattagaaacactcca

SEQ ID NO: 107 - DNA, 21 bp, Artificial Sequence
  SDF1 sense primer
ttgccagcacaaagacactcc

SEQ ID NO: 108 - DNA, 22 bp, Artificial Sequence
  SDF1 antisense primer
ctccaaagcaaaccgaatacag

SEQ ID NO: 109 - DNA, 20 bp, Artificial Sequence
  PDGFRa sense primer
atcaatcagcccagatggac

SEQ ID NO: 110 - DNA, 20 bp, Artificial Sequence
  PDGFRa antisense primer
ttcacgggcagaaaggtact

SEQ ID NO: 111 - DNA, 26 bp, Artificial Sequence
  VEGF sense primer
acatcttccaggagtaccctgatgag

SEQ ID NO: 112 - DNA, 22 bp, Artificial Sequence
  VEGF antisense primer
gcattcacatttgttgtgctgt

SEQ ID NO: 113 - DNA, 20 bp, Artificial Sequence
  RXR sense primer
acatggcttccttcaccaag

SEQ ID NO: 114 - DNA, 20 bp, Artificial Sequence
  RXR antisense primer
cagctcagcctccaggatcc

SEQ ID NO: 115 - DNA, 23 bp, Artificial Sequence
  Osteopontin sense primer
gctctagaatgagaattgcactg

SEQ ID NO: 116 - DNA, 20 bp, Artificial Sequence
  Osteopontin antisense primer
gtcaatggagtcctggctgt

SEQ ID NO: 117 - DNA, 18 bp, Artificial Sequence
  AP sense primer
tgaaatatgccctggagc

SEQ ID NO: 118 - DNA, 19 bp, Artificial Sequence
  AP antisense primer
tcacgttgttcctgtttag

SEQ ID NO: 119 - DNA, 20 bp, Artificial Sequence
  PPAR-gamma sense primer
aggcgagggcgatcttgaca

SEQ ID NO: 120 - DNA, 25 bp, Artificial Sequence
  PPAR-gamma antisense primer
accaggaatgcttttggcatactct

SEQ ID NO: 121 - DNA, 20 bp, Artificial Sequence
  Tuj sense primer
cctttggacacctattcagg

SEQ ID NO: 122 - DNA, 20 bp, Artificial Sequence
  Tuj antisense primer
gtgagtgtgtcagctggaag

SEQ ID NO: 123 - DNA, 21 bp, Artificial Sequence
  GFAP sense primer
tccgccaagccaagcacgaag

SEQ ID NO: 124 - DNA, 21 bp, Artificial Sequence
  GFAP antisense primer
catcccgcatctccacagtct

SEQ ID NO: 125 - DNA, 21 bp, Artificial Sequence
  Nestin sense primer
aactggcacacctcaagatgt

SEQ ID NO: 126 - DNA, 21 bp, Artificial Sequence
  Nestin antisense primer
tcaagggtattaccgaagggg

SEQ ID NO: 127 - DNA, 19 bp, Artificial Sequence
  MAP2 sense primer
tcagacttccaccgagcag

SEQ ID NO: 128 - DNA, 19 bp, Artificial Sequence
  MAP2 antisense primer
aggggaaagatcatggccc

(19) United States
(12) Patent Application Publication
(10) Pub. No.: US 2012/0003186 A1
(43) Pub. Date: Jan. 5, 2012

(54) METHOD FOR DEDIFFERENTIATING ADIPOSE TISSUE STROMAL CELLS

(75) Inventor: Soo-Kyung KANG, Seoul (KR)

(73) Assignee: SNU R&DB FOUNDATION, Seoul (KR)

(21) Appl. No.: 12/982,415

(22) Filed: Dec. 30, 2010

(30) Foreign Application Priority Data
Jun. 30, 2010 (KR) 10-2010-0062868

(51) Int. Cl. A61K 35/12; A61P 3/10; A61P 19/04; A61P 19/02; A61P 37/02; A61P 21/00; A61P 19/10; C12N 5/071; A61P 25/00

(52) U.S. Cl. 424/93.7; 435/377; 435/325

(57) ABSTRACT

A method for dedifferentiating adipose tissue stromal cells (ATSC) is provided. When the ATSC is treated under hypoxia condition and with a 4-(3,4-Dihydroxy-phenyl)-derivative (DHP-derivative), expression of stemness genes, cellular growth-related genes and cellular mobility-related genes increase, and expression of histone and DNA methylation-related genes decrease so that cell proliferation increases and pluripotency for differentiating into adipocytes, osteocytes, myocytes, beta cells and cartilage cells is acquired. When the dedifferentiated ATSC is implanted into animal model with spinal cord injury and diabetic animal model, effects of nerve regeneration and increased blood surge level are confirmed. As a result, the method for dedifferentiating the ATSC can be effectively used in the stem cell research, tissue regeneration and development of cytotherapeutic medicines.

### CROSS-REFERENCES TO RELATED APPLICATIONS

This patent application claims the benefit of priority from Korean Patent Application No. 10-2010-0062868, filed on Jun. 30, 2010, the contents of which are incorporated herein by reference in its entirety.

### BACKGROUND OF THE INVENTION

1. Field of the Invention

The invention relates to a method for dedifferentiating somatic cells.

2. Description of the Related Art

Although the classic definition of cell plasticity from stem cell biology specifies the ability of stem cells to differentiate into a variety of cell lineages, the term is also currently applied to the ability of a given cell type to reciprocally dedifferentiate, re-differentiate, and/or trans-differentiate in response to specific stimuli (Goodell M A et al., 2003, Curr opin hematol., 10: 208-13; Wagers A J et al., 2004, Cell, 116: 639-48). Cellular de-differentiation underlies contemporary topical issues in stem cell biology, most notably regeneration and nuclear cloning. In stem cell biology, this process characterizes the transition of differentiated somatic cells to pluripotent stem cells, and is accompanied by global chromatin reorganization, which is itself associated with the reprogramming of gene expression. De-differentiation signifies the withdrawal of cells from a given differentiated state into a stem cell-like state, which confers pluripotency, a process that precedes re-entry into the cell cycle (Grafi G et al., 2004, Dev Biol., 268: 16). The state of de-differentiation can be determined by changes in cell morphology, genome organization, and the gene expression pattern, as well as by the capability of protoplasts to differentiate into multiple types of cells, depending on the type of applied stimulus (Takebe I et al., 1971, Naturwissenschaften., 58: 318320; Valente O et al., 1998, Plant Sci., 134: 207215; Zhao J et al., 2001, J Biol. Chem., 276: 2277222778; Avivi Y et al., 2004, Dev Dyn., 230: 1222). Histone methylation activity is required for the establishment and maintenance of the dedifferentiated state and/or re-entry into the cell cycle. The complexity of cellular de-differentiation, and particularly the occurrence of DNA recombination, can result in genome instability (Grafi G et al., 2007, Dev Biol., 15; 306(2):838-46). Several studies have demonstrated that freezing-induced and traumatic CNS-induced injuries facilitate the appearance of some radial glia-like fibers, which express Nestin in adult rodents (Hatten M E et al., 1984, Brain Res., 315: 309313; Rosen G D et al., 1992, J. Neuropathol Exp Neurol., 51: 601611; Rosen G D et al., 1994, Brain Res Dev., 82:127135; Hunter K E et al., 1995, Proc Natl Acad Sci USA, 92: 20612065; Shibuya S et al., 2002, Neuroscience, 114: 905916; Huttmann K et al., 2003, Eur J Neurosci., 18: 27692778). A variety of transitional forms of cells are observed during transformation from radial glia to astroglia in vivo (Pixley S K et al., 1984, Brain Res., 317: 201209; Hartfuss Et al., 2001, Dev Biol., 229:1530; Alves J A et al., 2002, J Neurobiol., 52: 251265). These experimental results provide a simple means for the acquisition of sizeable quantities of immature stem cells from the de-differentiation of mature cells. Stem and/or precursor cells exist within a distinct tissue structure referred to as the niche, which regulates their self-renewal and differentiation (Eckfeldt C E et al., 2005, Nat Rev Mol Cell Biol., 6: 726-737; Ceradini D J et al., 2004, Nat. Med., 10: 858-864). As recently demonstrated, the bone marrow microenvironment has a lower oxygen concentration than other tissues, and stem cells are localized within the hypoxic regions (Ezashi T et al., 2005, Proc Natl Acad Sci USA, 102: 4783-4788). thereby indicating that hypoxia may be crucial for the maintenance of stem cells. Under hypoxic conditions, the differentiation of embryonic stem cells, as well as precursor cells, is inhibited (Yun Z et al., 2002, Dev. Cell, 2: 331-341; Yaccoby, S et al., 2005, Clin cancer Res., 11(21): 7599-7606; Kang S K et al., 2003, Exp Neurol., 183(2): 355-66). Conversely, the pro-differentiation gene is also downregulated as a result of HIF1α activation (Kang S K et al., 2004, J Cell Sci., 117: 4289-99). Recent observations have demonstrated that adult somatic stem cells have the capacity to participate in the regeneration of different tissues (Akimoto T et al., 2004, Int J Radiat Biol., 80: 483-492; NG D C et al., 2000, J Biol. Chem., 275: 40856-40866), thereby suggesting that restrictions on differentiation are not completely irreversible and can be reprogrammed with de-differentiation and trans-differentiation processes.

In our continuous efforts to find the explanation about the characteristics of the dedifferentiation, the inventors have developed a well-defined biological system involving human adipose tissue stromal cells (ATSC) with the potential to differentiate into multiple cell lineages, including neurons with active migration activity (NG D C et al., 2000, J Biol Chem., 275: 40856-40866). The inventors continued research to find a method for dedifferentiating ATSC into stem cells to even more early stage, and found that, by hypoxia/DHP-derivative [4-(3,4-dihydroxy-phenyl)-derivative] treatment, it is possible to dedifferentiate ATSC into stem cells with even higher cellular proliferation ability and pluripotency, and confirmed the effects of dedifferentiated ATSC of neural regeneration and increase of blood sugar levels in the animal models with nerve injuries and diabetic animal models. Therefore, the inventors completed the present invention by confirming that the dedifferentiating method can be effectively used in stem cell researches, regeneration of tissues and development of cytotherapeutic medicines.

### SUMMARY OF THE INVENTION

One object of the present invention is to provide a composition which induces dedifferentiation of differentiated cells.

Another object of the present invention is to provide a method for inducing dedifferentiation of differentiated cells using said composition.

In order to accomplish the above-mentioned objects of the present invention, a composition for dedifferentiating differentiated cells into pluripotent stem cells is provided, in which the composition includes 4-(3,4-Dihydroxy phenyl)-derivative (DHP-derivative) or pharmaceutically-acceptable salt thereof as an effective ingredient.

In one embodiment, a method for inducing dedifferentiating of cells is also provided, including the steps of: i) culturing differentiated cells to induce differentiation; and ii) culturing the cultured cells in a culture medium containing DHP-derivative or pharmaceutically-acceptable salt thereof under hypoxia condition.

It was confirmed that when the differentiated cells or adipose tissue stromal cells (ATSC) are exposed to the hypoxia condition and treated with 4-(3,4-dihydroxy phenyl)-derivative (DHP-derivative) for dedifferentiation, expressions of the stemness genes, cellular growth-related genes and cellular mobility-related genes increase, expressions of histone and DNA methylation-related genes decreases, cellular proliferation increase, and characteristics of pluripotent stem cell which can differentiate into adipose cells, osteocyte, myocyte, beta cells and cartilage cells appeared. Additionally, when the differentiated cells or ATSC, which were dedifferentiated according to an embodiment of the present invention, were implanted into animal models with spinal damages and diabetic animal models, the effects of nerve regeneration and increased blood sugar level were confirmed. As a result, a method of dedifferentiating ATSC according to an embodiment of the present invention can be effectively used in stem cell research, tissue regeneration and for the development of cytotherapeutic medicines.

### BRIEF DESCRIPTION OF THE DRAWINGS

The above and/or other aspects of what is described herein will be more apparent by describing certain exemplary embodiments with reference to the accompanying drawings, in which:

FIG. 1a shows the result of analyzing percentage of ATSC with hypoxia/DHP-derivative treatment (experimental group) or ATSC with DHP-derivative treatment (control group) at stages G0/G1, S and G2/M of cell cycles, respectively;

FIG. 1b shows the result of confirming cellular proliferation through colony forming test;

FIG. 1c shows the result of confirming an amount of telomerase in accordance with telomerase activity by PCR enzyme-linked immunoabsorbent assay;

FIG. 2a show the result of confirming changes in expression of epitope on cell surface in response to ATSC dedifferentiation by hypoxia/DHP-derivative treatment;

FIG. 2b show the result of confirming FACS analysis of the expression of stemness marker proteins;

FIG. 3a shows the result of confirming expressions of stemness, nerve marker, cellular proliferation-related genes and proteins in response to ATSC dedifferentiation by hypoxia/DHP-derivative treatment by real-time RT-PCR;

FIG. 3b shows the result of FIG. 3a by Western blotting;

FIG. 4 shows the result of analyzing a karyotype of experimental cell group and control cell group;

FIG. 5a shows the result of examining changes in cell morphology of completely-differentiated cartilage cells and adipose cells, and cartilage cells and adipose cells with hypoxia/DHP-derivative, under a microscope respectively;

FIG. 5b shows the result of confirming cellular proliferation by colony forming test;

FIG. 5c shows the result of confirming expressions of stemness genes and proteins by real-time RT-PCR and Western blotting, respectively;

FIG. 6 shows the result of analyzing, by functions, the genes of the dedifferentiated ATSC expressed two times or more that those of the ATSC, based on the examination on the gene arrays of the dedifferentiated ATSC and the ATSC, respectively;

FIG. 7a shows the result of analyzing a difference between genes expressed in ATSC and hESD, and genes expressed in dedifferentiated ATSC and hESC by gene array analysis;

FIG. 7b shows the result of confirming by RT-PCR an amount of expression of embryonic genes (Utf1, Dapp5, FGF4 and ERas) from among the genes which were not expressed in ATSC but expressed in dedifferentiated ATSC and hESC;

FIG. 8 shows the result of observing differences of methylation patterns between experimental and control cell groups;

FIG. 9a shows the result of confirming by Western blotting the expression pattern of growth-related signal genes and Rex-1 in ATSC treated with hypoxia/DHP-derivative;

FIG. 9b shows the result of FIG. 9a by real-time PCR;

FIG. 10 shows the result of colony forming test of the cell growth of ATSC treated with hypoxia/DHP-derivative;

FIG. 11a shows the result of observing expression of stemness genes and cellular proliferation-related genes and changes in cellular proliferation after cells are treated with siRNA, with respect to REX1 of dedifferentiated ATSC;

FIG. 11b shows the result of FIG. 11a with respect to Oct4 of dedifferentiated ATSC;

FIG. 11c shows the result of FIG. 11a with respect to HIF1 α of dedifferentiated ATSC;

FIG. 12 shows a signal pathway to regulate expressions of intranuclear Rex1, Nanog, p53, p21 and c-myc genes, and expression of stemness genes such as Rex1, Sox2, Oct4 and Klf4 (Kruppel-like factor 4) by HIF1 α;

FIG. 13 shows the result of confirming in vitro migration activity of ATSC by migration assay and confirming cell migration-related genes by RT-PCR, respectively;

FIG. 14 shows the result of confirming possible relationship between in vitro migration activity of ATSC treated with hypoxia/DHC-derivative and the duration of hypoxia stimulus treatment, based on phosphorylation of cell migration-related proteins by Western blotting;

FIG. 15 shows the result of testing cell migration in the control cell group of signal pathway influencing the in vitro migration of ATSC treated with hypoxia/DHP-derivative, and experimental cell group either treated with PD98059 or SB203580, or not treated at all;

FIG. 16 shows the signal pathway to regulate the cell migration of dedifferentiated ATSC;

FIG. 17 shows the result of confirming re-differentiation of the dedifferentiated ATSC into osteocyte or adipose cells, by Alzarin red dye (osteocyte) and Oil Red O dye (adipose cells), and RT-PCR analysis of osteocyte differentiation-related transcription factor;

FIG. 18 shows the result of observing re-differentiation of the dedifferentiated ATSC in animal model into osteocyte (Alzarin Red dye), cartilage cells (Masson dye) or myocyte (Van Gieson);

FIG. 19 show the result of confirming whether or not teratoma was formed in germline-derived tissue or organs (muscle, nerve, pigment cells, adipose cell and secreting gland) in a mouse implanted with ATSC or dedifferentiated ATSC;

FIG. 20a show the results of confirming if dedifferentiated ATSC differentiates in vitro into neuron by immunocytochemical staining and Western blotting, respectively;

FIG. 20b shows the result of FIG. 20a by RT-PCR;

FIG. 21a shows the result of measuring BBB (Basso, Beattie and Bresnahan) scores for 6 weeks in spinal cord injury (SCI) rat animal model implanted with ATSC or dedifferentiated ATSC;

FIG. 21b shows the result of confirming differentiation of neuron by immunicytochemical staining;

FIG. 21c shows the result of measuring evoked action potential of trans-differentiated neuron prior to sciatic axotomy, immediately after sciatic axotomy, and 30 days after sciatic axotomy, respectively;

FIG. 22a shows the result of observing beta cell differentiation ability of dedifferentiated ATSC by immunocytochemical staining and Western blotting;

FIG. 22b shows the result of confirming by Western blotting expression of insulin and c-peptide proteins in experimental cell group and control cell group which are differentiated into beta cells;

FIG. 23a shows the result of observing regeneration of tissues of pancreatic islets in normal mouse, diabetic mouse and diabetic mouse implanted with ATSC or dedifferentiated ATSC, respectively, by immunocytochemical staining;

FIG. 23b shows the ratio of insulin (+) cells of each mouse with respect to normal mouse from the result of FIG. 23a; and

FIG. 24 shows the result of measuring the blood sugar level of diabetic mouse implanted with ATSC or dedifferentiated ATSC.

### DETAILED DESCRIPTION OF EXEMPLARY EMBODIMENTS

Features and advantages of the present invention will be more clearly understood by the following detailed description of the present preferred embodiments by reference to the accompanying drawings. It is first noted that terms or words used herein should be construed as meanings or concepts corresponding with the technical spirit of the present invention, based on the principle that the inventors can appropriately define the concepts of the terms to best describe their own invention. Also, it should be understood that detailed descriptions of well-known functions and structures related to the present invention will be omitted so as not to unnecessarily obscure the important point of the present invention.

The present invention will now be described in further detail by examples. It would be obvious to those skilled in the art that these examples are intended to be more concretely illustrative and the scope of the present invention as set forth in the appended claims is not limited to or by the examples.

In one embodiment, a composition to dedifferentiate differentiated stem cells into pluripotent stem cells is provided, in which the composition includes 4-(3,4-dihydroxy-phenyl)-derivative (DHP-derivative) or pharmaceutically-acceptable salt thereof as an effective ingredient.

The ‘differentiated cells’ herein may be adiopose tissue stromal cells (ATSC), adipose stromal cells (ASC), or differentiated somatic cells, and the differentiated somatic cells may be osteocytes or adipocytes.

A purified DHP-derivative was provided by Dr. Ho-Gon CHUN of the Korea Research Institute of Bioscience and Biotechnology. To be specific, the DHP-derivative was purified from pellinus linteus as described in the thesis of Min-Ki JEE [Jee M K et al., 2010, PLoS ONE 5(2): e9026]. ATSC with stem cell activity was collected by the known treatment from unprocessed donor-derived adipose tissue [Kang S K et al., 2003, Exp Neurol., 183(2): 355-66; Kang S K et al., 2004, J Cell Sci., 117: 4289-99], and used. The inventors cultured the ATSC in culture medium containing DHP-derivative and under hypoxia condition to dedifferentiate the ATSC and collected the cultured ATSC.

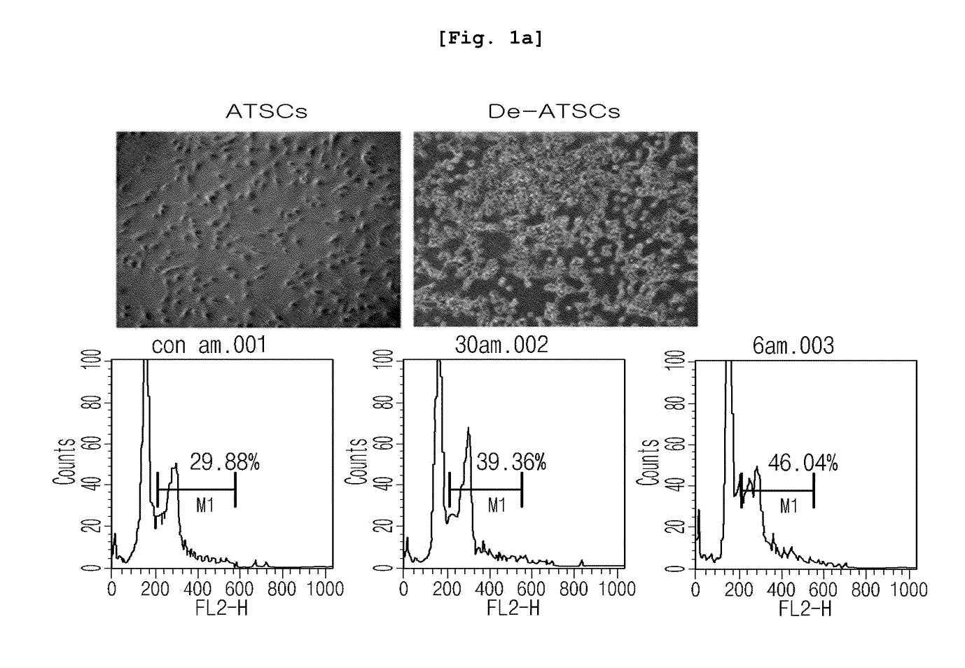

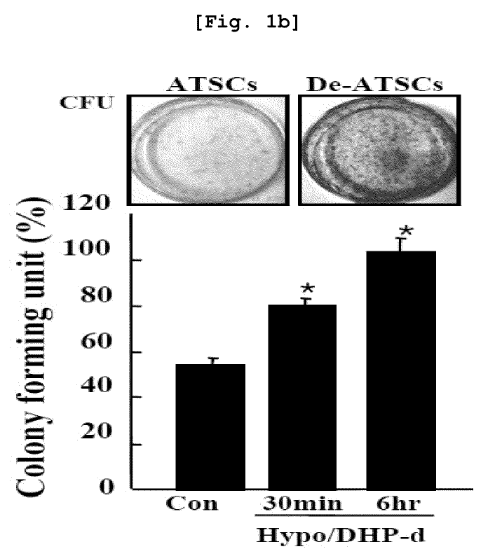

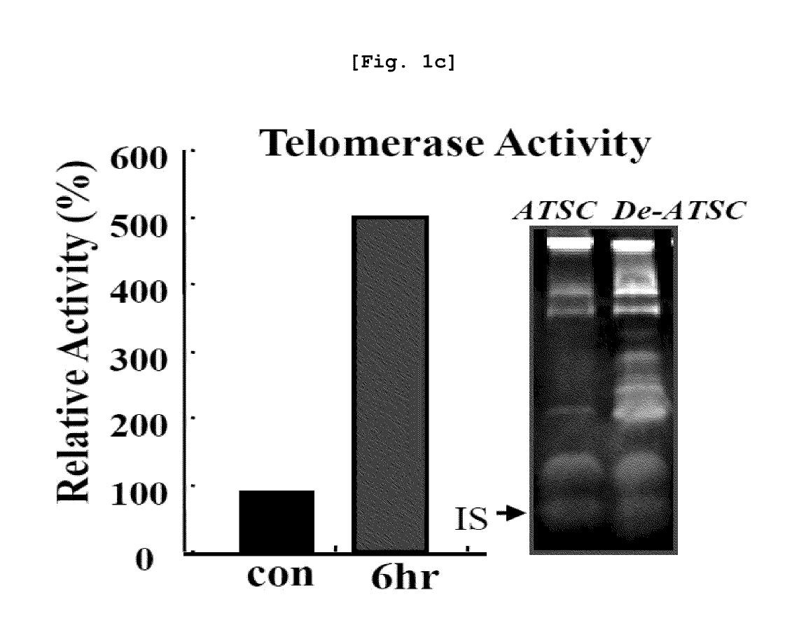

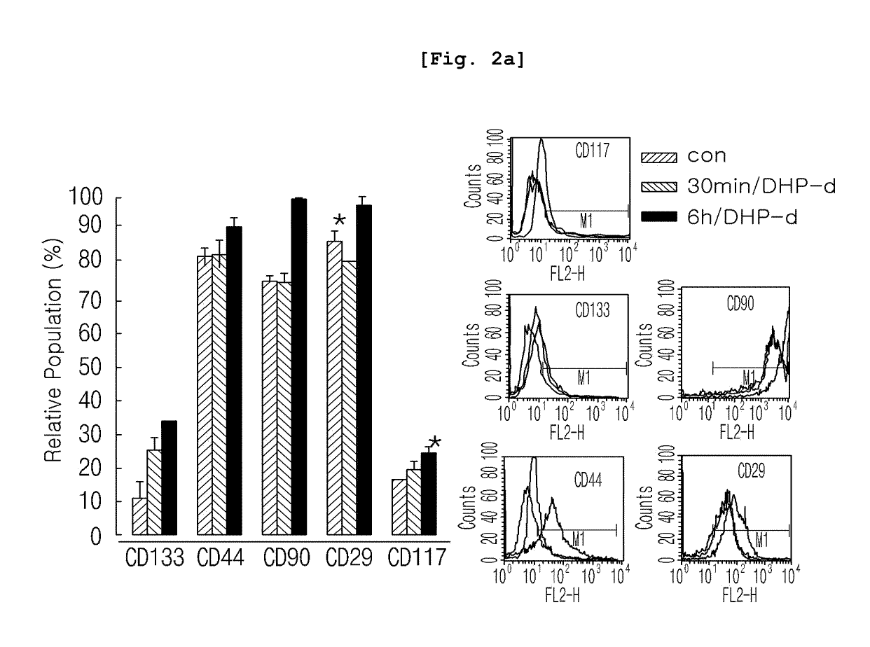

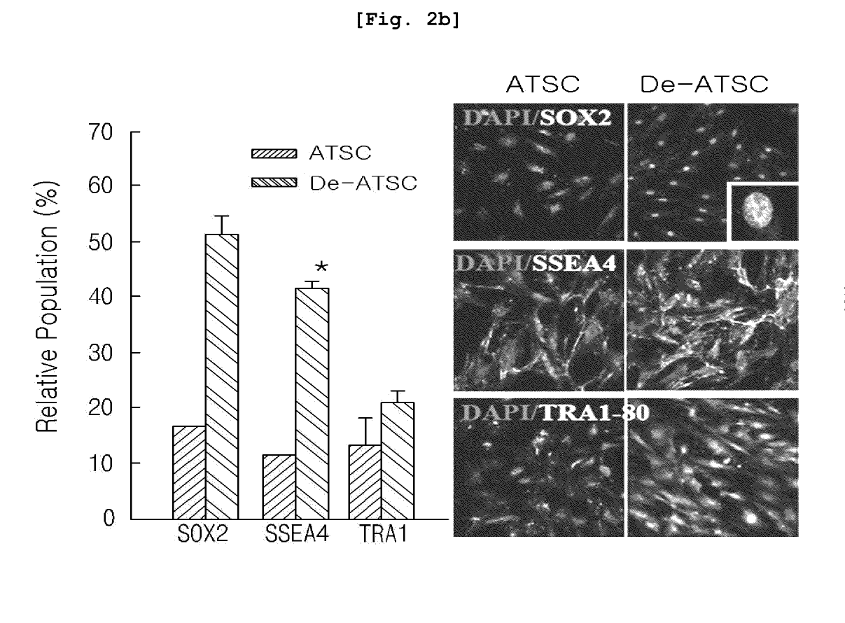

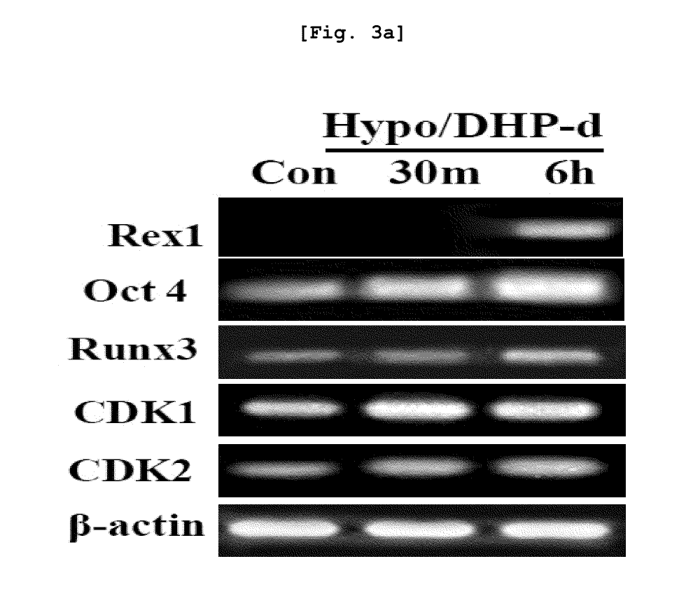

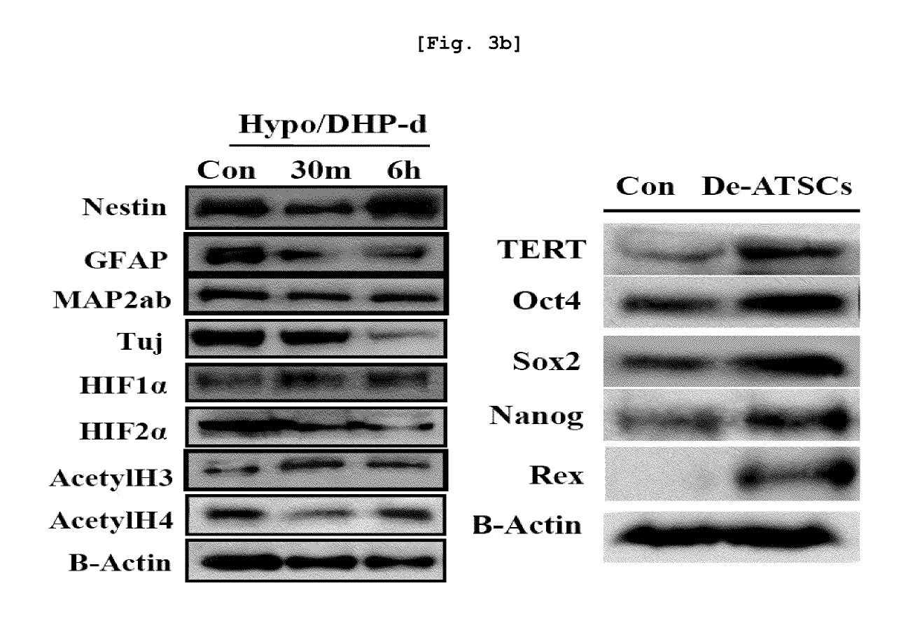

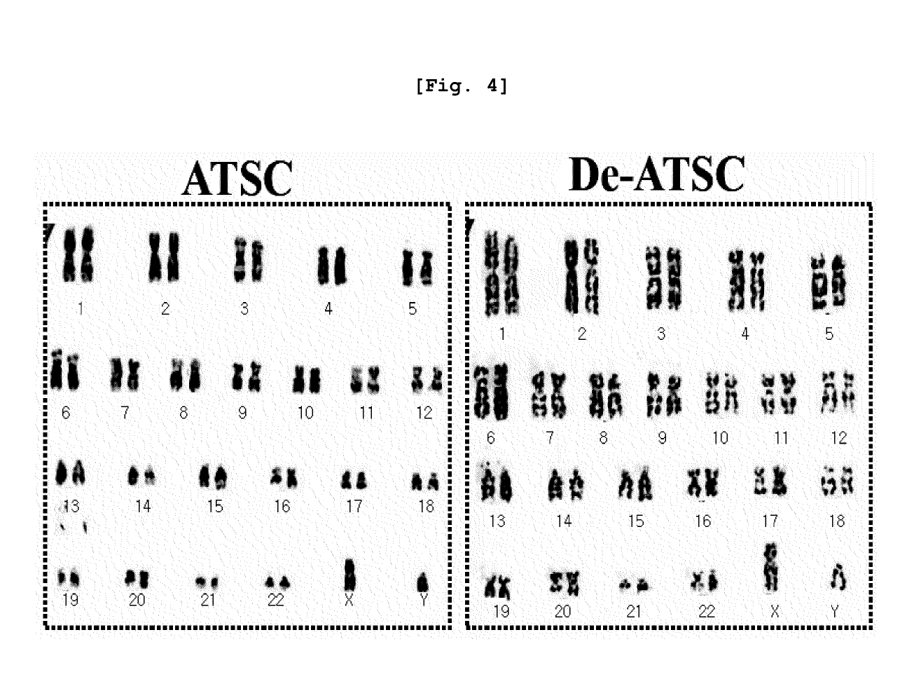

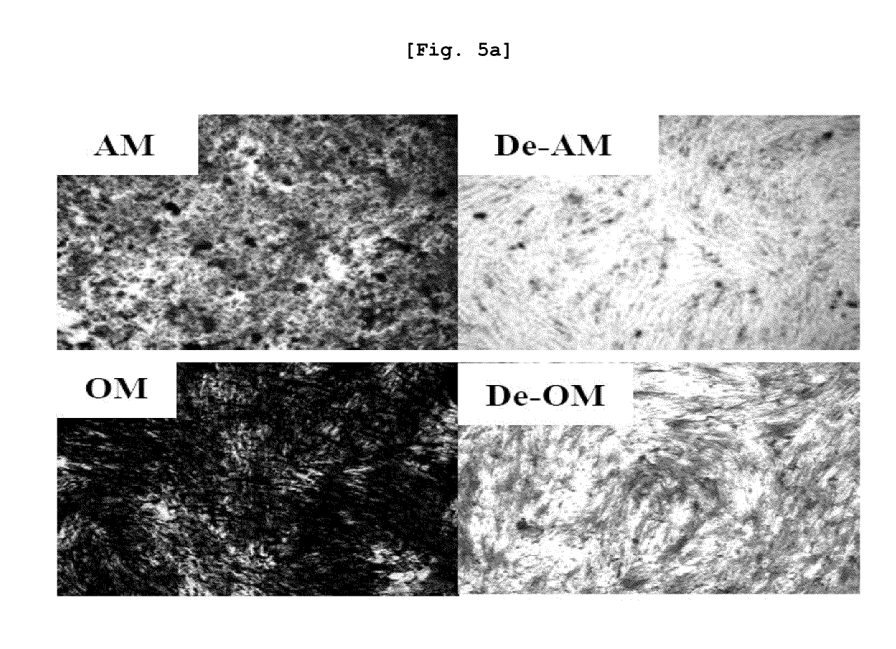

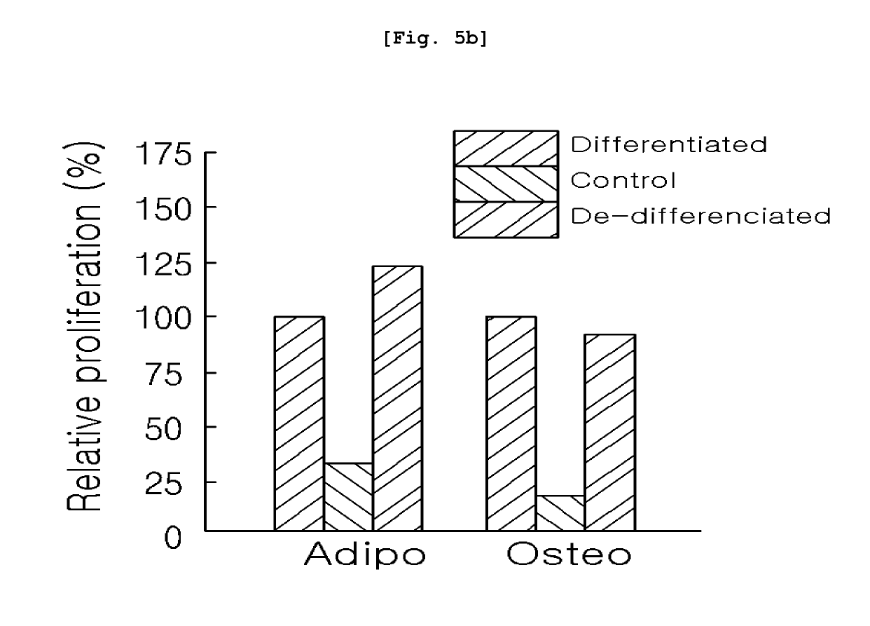

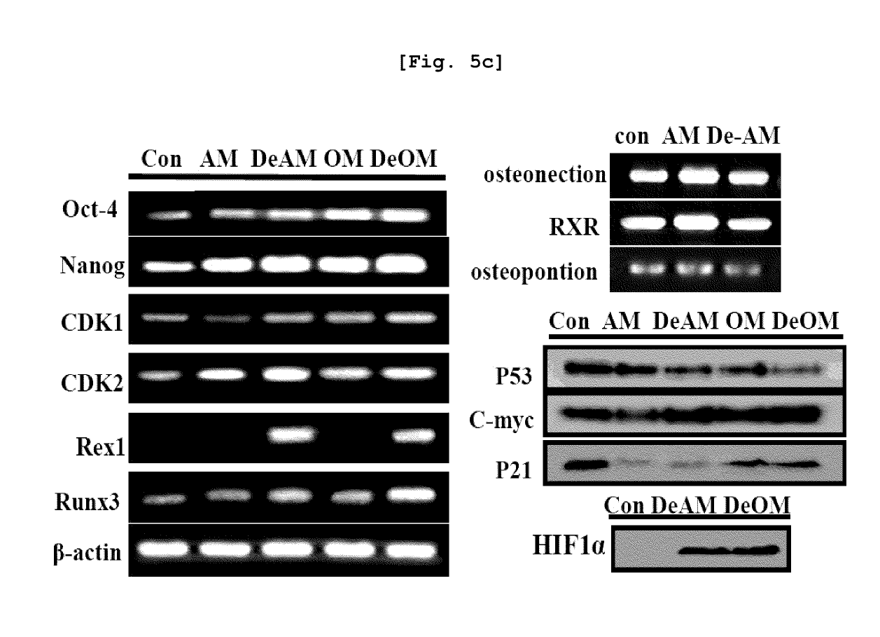

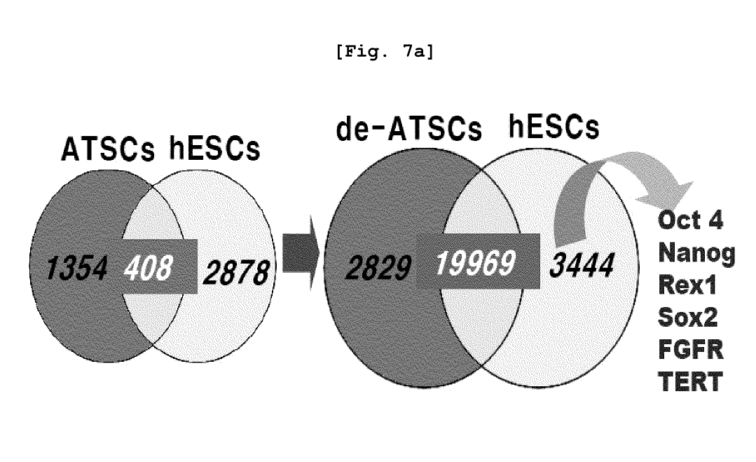

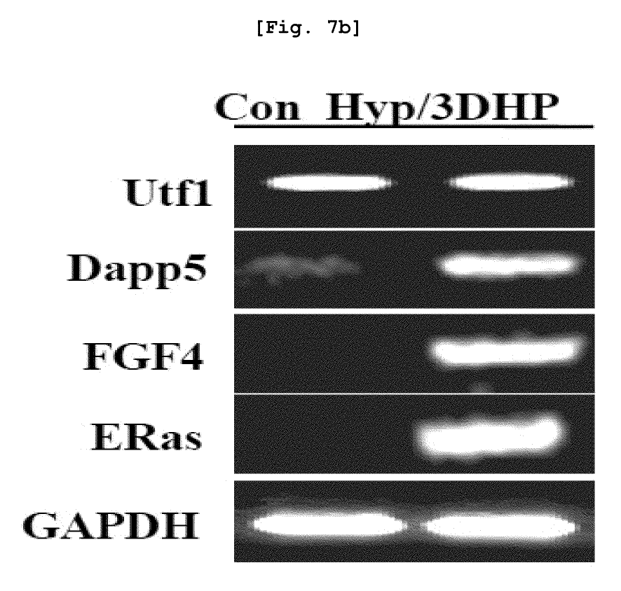

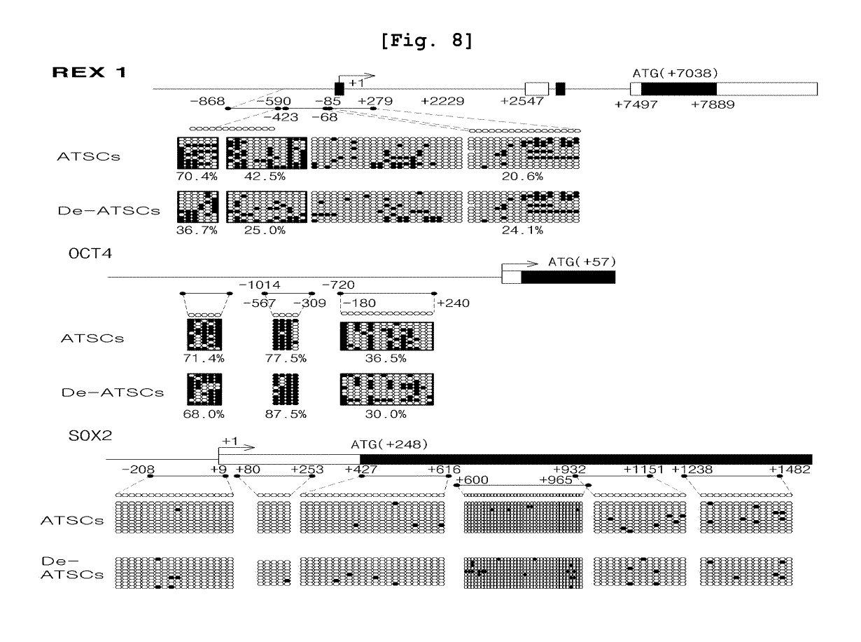

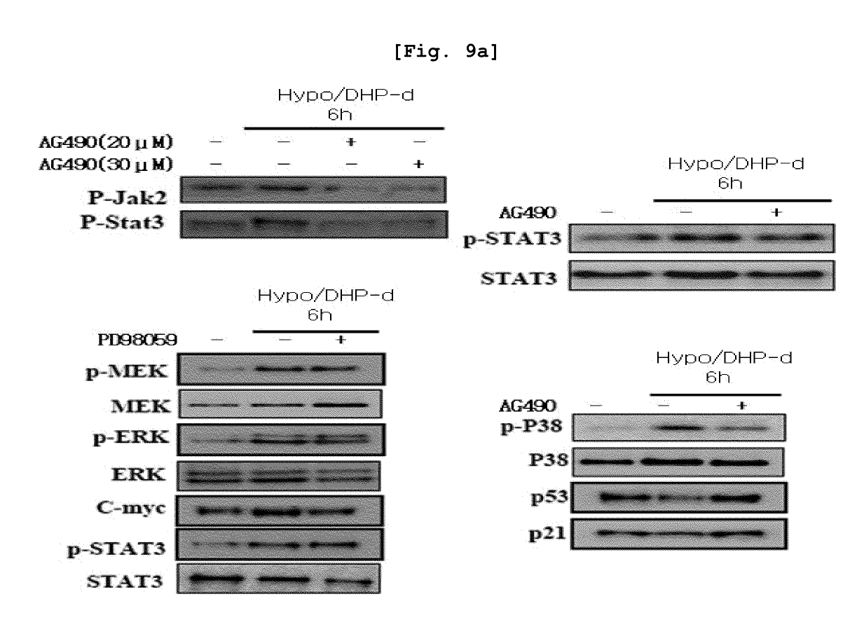

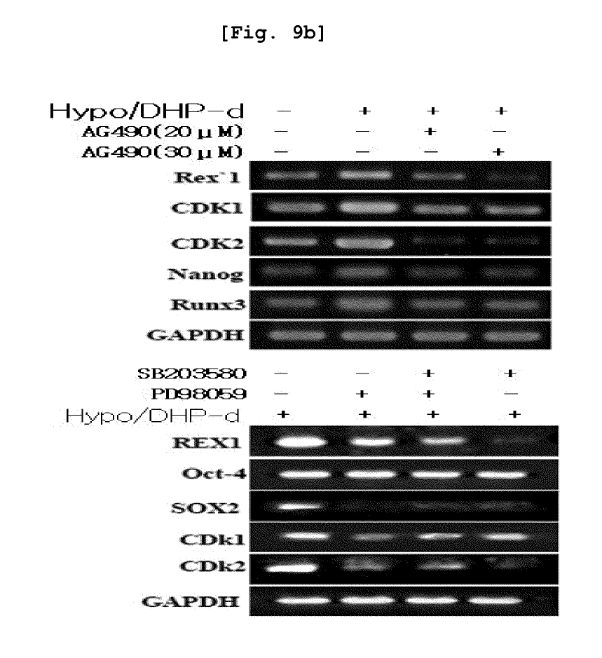

As a result of comparing the ATSC (i.e., experimental group) which were dedifferentiated with hypoxia/DHP-derivative treatment, with ATSC (i.e., control group) which were treated and cultured with DHP-derivative but under normal oxygen condition, it was confirmed that the experimental group showed higher cellular proliferation ability, colony forming ability and telomerase activity, and thus had more active cell cycle (FIGS. 1a, 1b and 1c). Also, as a result of confirming the stem cell marker proteins of the experimental group and the control group by immunocytochemical analysis, it was confirmed that the experimental group exhibited more expression (FIGS. 2a and 2b). Furthermore, the experimental group exhibited relatively more expression of stemness genes, nerve markers, cellular proliferation-related genes and proteins compared to the control group (FIGS. 3a and 3b). It was also confirmed that the experimental group had normal chromosome, showing that the group included dedifferentiated stem cells, and not cancer cells (FIG. 4). With the dedifferentiation according to an embodiment of the present invention, it was confirmed that the fully-differentiated adipocytes and osteocytes can also be dedifferentiated (FIGS. 5a, 5b and 5c). Additionally, from the fact that HIF1 α (hypoxia-inducible factor 1 α) is not expressed in ATSC but expressed in dedifferentiated adipocytes and osteocytes, culturing under hypoxia condition is important for cellular dedifferentiation. As a result of comparing the patterns of gene expressions in dedifferentiated adipocytes and osteocytes of experimental group and control group with each other based on the patterns of gene expressions in human embryonic stem cell (hESC) and microarray, it was confirmed that the gene expression pattern of the experimental group is more similar to hESC (FIG. 7a). Additionally, as a result of checking whether or not the promoter of the stemness gene underwent methylation in the experimental group and the control group, respectively, it was confirmed that methylation decreased on promoter regions of REX1 (Zinc Finger Protein 42; ZFP42) and OCT4 (Octamer-4) (FIG. 8). This suggests that methylation of stemness genes decreased due to hypoxia/DHP-derivative treatment and thus gene expression increased. Further, as a result of checking a signal pathway of a gene involved in growth-related signal transmission in the experimental group and the control group by checking whether or not the protein are activated, an amount of expression, and with inhibitor treatment and knockdown method using siRNA, it was confirmed that proliferation of the dedifferentiated ATSC increased by JAK/STAT3 (Janus kinase 3/signal transducer and activator of transcription) and MEK (Mitogen-activated Protein/ERK Kinase) signal pathway (FIGS. 9a, 9b, 10, 11a, 11b, 11c and 12).

The ‘hypoxia’ condition herein refers to a condition where there is a low amount of oxygen below a normal oxygen content. The oxygen may preferably range between 0.1% and 15%, and more preferably between 0.5% and 5%, and yet more preferably be at 1%.

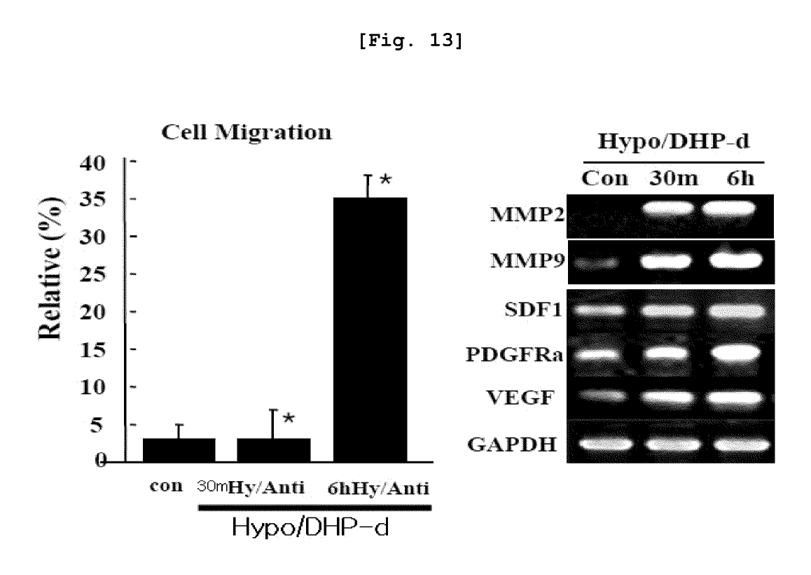

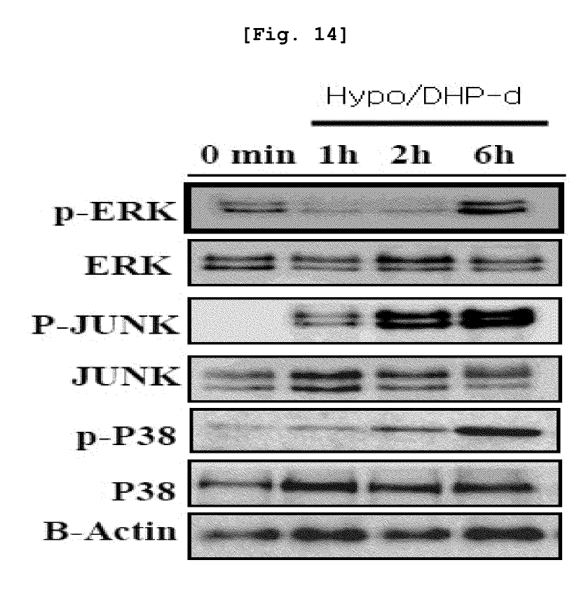

In order for the dedifferentiated cell with pluripotency to differentiate normally, cell mobility is considered to be important. The present inventors confirmed that the experimental group in vitro showed significantly higher mobility than the control group (FIG. 13), and that such increased cell mobility was attributable to the increase of activity and expression of cellular migration-related proteins (FIGS. 14, 15 and 16).

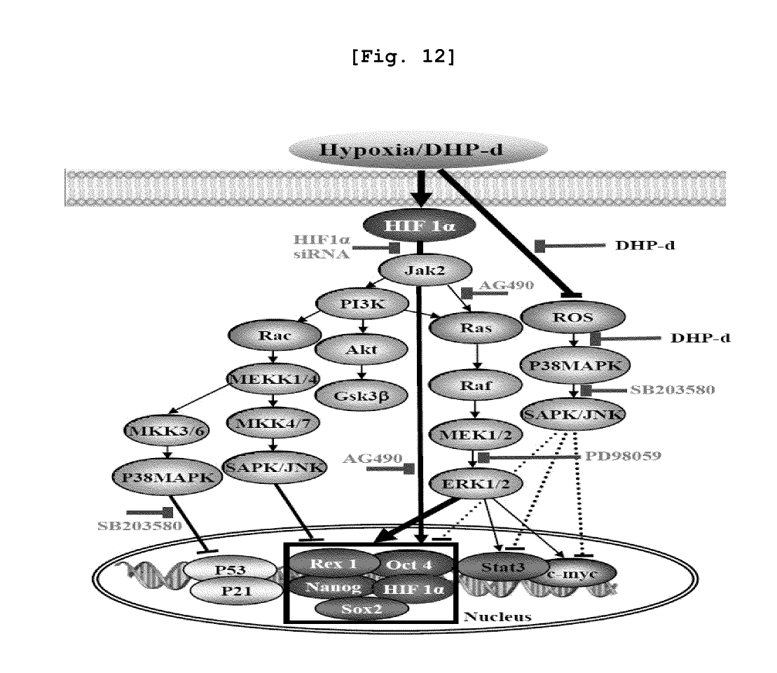

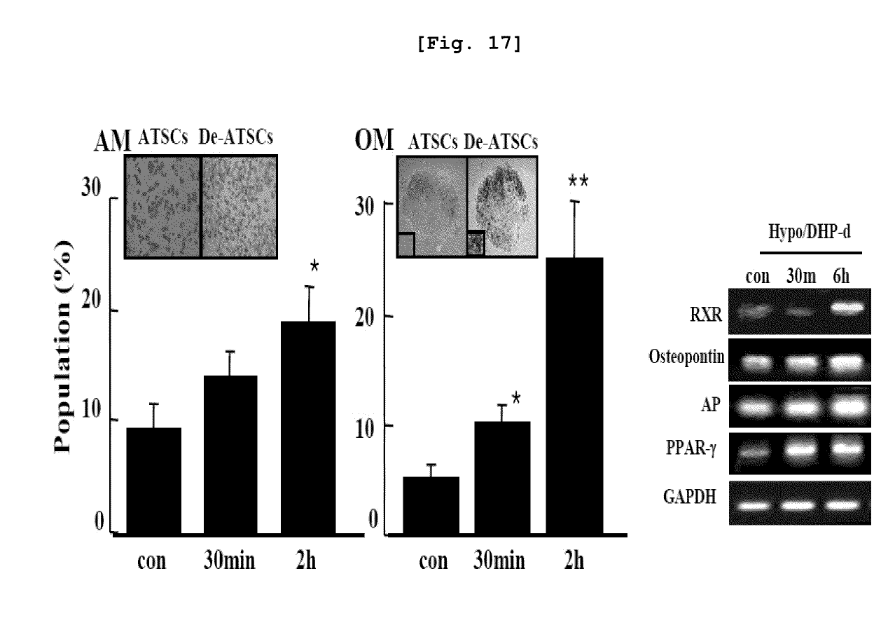

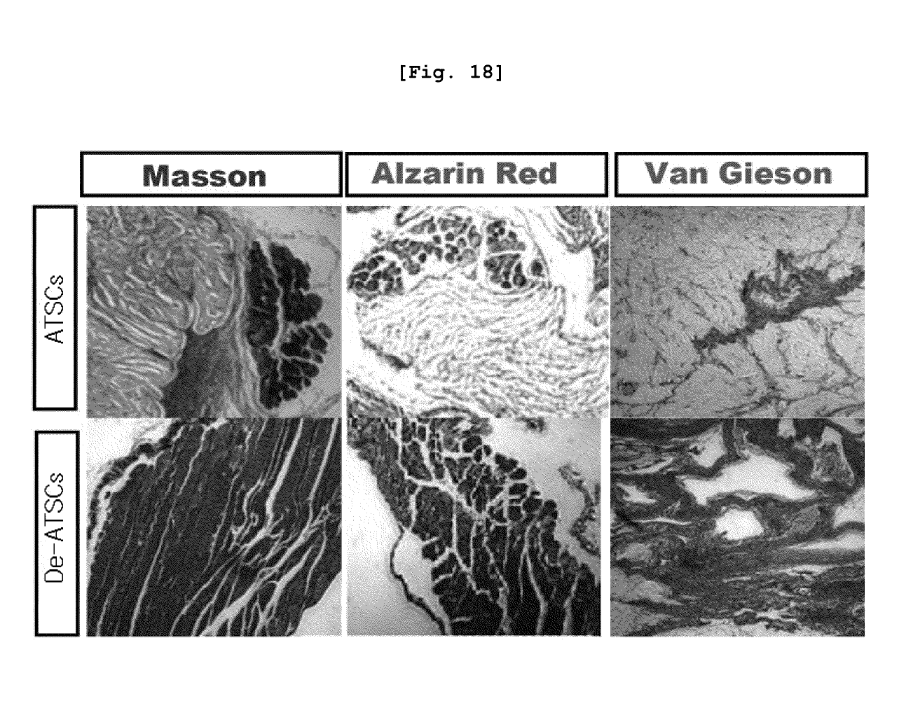

As a result of re-differentiating to prove the pluripotency of the dedifferentiated ATSC, it was confirmed that the experimental group in vitro underwent differentiation into adipocytes, osteocytes, neurons and beta cells with higher efficiency than the control group (FIGS. 17, 18, 19, 22a and 22b), and that the experimental group in vivo underwent differentiation into neurons, myocyte, osteocyte, cartilage cells, pancreatic islet, pigment cells, and secretory organ (FIGS. 18, 19, 21b, 23a and 23b). Additionally, as a result of implanting the cells of the experimental group into nerve-damaged animal models and diabetic animal models, treatment effect was confirmed (FIGS. 12a, 21c and 24).

Accordingly, since it is confirmed that the ATSC are dedifferentiated into stem cells of very early stage with the treatment with a composition containing DHP-derivative according to an embodiment of the present invention, the DHP-derivative or pharmaceutically-acceptable salt thereof according to an embodiment of the present invention can be used for the dedifferentiation of the cells.

The DHP-derivatives are compounds represented by chemical formula 1 below, wherein R is one selected from a group consisting of hydrogen and C₁˜C₄ alkyl group, and preferably the DHP-derivative is a compound represented by chemical formula 2 below.

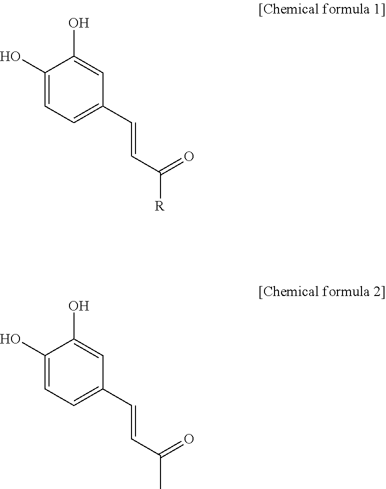

The DHP-derivative according to an embodiment of the present invention may be extracted from phellinus linteus. For extraction, phellinus linteus, naturally-grown, farmed or purchased, may be used, and extraction method using an extracting device such as supercritical fluid extraction, critical fluid extraction, high temperature extraction, high pressure extraction or ultrasound wave extraction may be used. Additionally, any conventionally-known extraction methods, such as using an adsorptive resin containing XAD and HP-20, may be used for the extraction. Reflux extraction is preferably carried out at elevated temperature or atmospheric temperature, but not strictly limited thereto. The extraction may be carried out preferably from 1 to 5 times, or 3 times more preferably, but not strictly limited thereto.

The phellinus linteus extract may preferably be obtained by extracting dried forms of each plant with water, C₁˜C₄ alcohol, or solvent blending the two, or more preferably, with C₁˜C₄ alcohol, or most preferably, with methanol or ethanol. By way of example, dried phellinus linteus may be grinded to fragments of appropriate size, put into an extraction receptacle, added with low C₁˜C₄ alcohols or solvent blending the same, or preferably, with methanol or ethanol, and then boiled, refluxed and extracted, and left for a predetermined time, filtered through a filter paper or the like. As a result, alcohol extract can be obtained. The extraction time may preferably range between 2 and 12 hours, and most preferably between 3 and 5 hours. After that, concentration or lyophilization may additionally be carried out.

Meanwhile, the phellinus linteus fractions according to an embodiment of the present invention may be n-hexane fractions and ethylacetate fractions obtained by dividing the extract with n-hexane and ethylacetate in sequence.

The composition according to an embodiment of the present invention may be a culture medium containing a DHP-derivative or pharmaceutically-acceptable salt thereof in an amount of 0.1 to 100 ng/ml, or preferably a culture medium containing a DHP-derivative or pharmaceutically-acceptable salt thereof in an amount of 1 to 50 ng/ml, or more preferably, a culture medium containing a DHP-derivative or pharmaceutically-acceptable salt thereof in an amount of 10 ng/ml, but not strictly limited thereto.

Furthermore, according to an embodiment, a method for inducing dedifferentiation of cells may include the steps of: 1) culturing differentiated cells to induce differentiation; and 2) culturing the cultured cells in a culture medium containing DHP-derivative or pharmaceutically-acceptable salt thereof.

Additionally, a step of culturing the cultured cell of step 2) under a culture condition of dedifferentiated cells may be provided.

The differentiated cells may be adiopose tissue stromal cells (ATSC), adipose stromal cells (ASC), or differentiated somatic cells, and the differentiated somatic cells may be osteocyte or adipocytes. The differentiated cell may be, most preferably, ATSC.

The ATSC, which are dedifferentiated according to an embodiment of the present invention, exhibited increased expression of stemness genes, cell growth-related genes, and cell migration-related genes, decreased expression of histone and DNA methylation-related genes, and increased cellular proliferation, and pluripotency to differentiate into adipocytes, osteocyte, myocyte, beta cells, neurons and cartilage cells. Additionally, the dedifferentiated ATSC according to an embodiment of the present invention, when implanted in animal models with spinal cord injuries and animal models with diabetes, exhibited improvement of lesions. Therefore, a ATSC dedifferentiation method according to an embodiment of the present invention can be effectively used in the stem cell research, tissue regeneration and development of cytotherapeutic medicines.

The culture medium of step 2) may include DHP-derivative or pharmaceutically-acceptable salt thereof in an amount of 0.1 to 100 ng/ml, or preferably, 1 to 50 ng/ml, or more preferably, 10 ng/ml, but not strictly limited thereto.

The culture condition for dedifferentiated ATSC of the additional step may include content of DPH-derivative or pharmaceutically-acceptable salt thereof in a amount of 0.1 to 100 ng/ml, or preferably, 1 to 50 ng/ml, or more preferably 10 ng/ml, and 37° C. and 5% CO₂, but not strictly limited thereto.

The hypoxia condition of step 2) may preferably include oxygen content in a range of 0.1 to 15%, or preferably 0.5% to 5%, or most preferably 1%.

Furthermore, the condition at step 2) may include that the ATSC is in gaseous state including 0.1% to 15% oxygen, 5% carbon dioxide, and 80% to 94.9% nitrogen, or preferably, 0.5 to 5% oxygen, 5% carbon dioxide, and 90% to 94.5% nitrogen, or more preferably, 1% oxygen, 5% carbon dioxide, and 90% to 94.5% nitrogen, but not strictly limited thereto.

Furthermore, in one embodiment, stem cells, which are dedifferentiated to have pluripotency by said differentiation method, are provided.

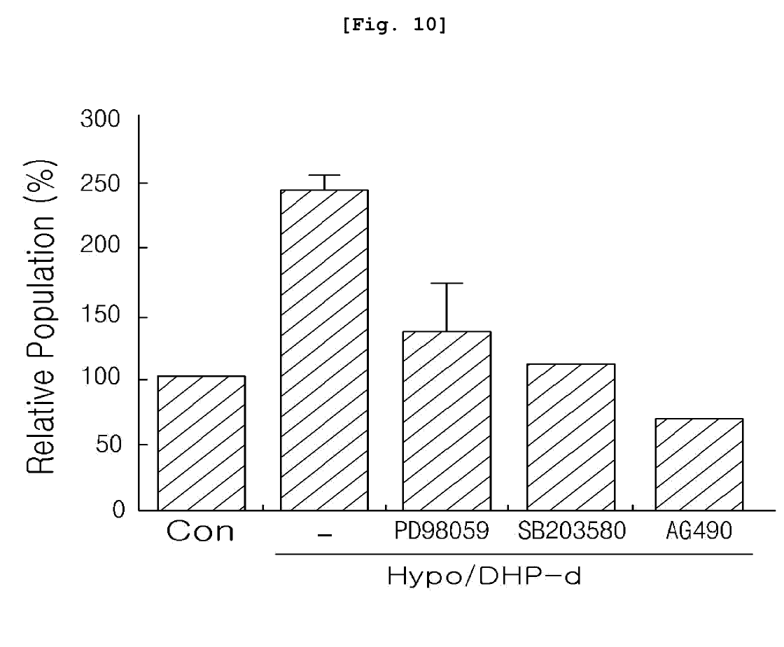

It was confirmed that the pluripotent dedifferentiated stem cells showed more active differentiation compared to the stem cell before dedifferentiation. It was particularly confirmed that the dedifferentiated ATSC exhibited superior re-differentiation ability compared to the pluripotent stem cell (FIG. 10), and as a result, the pluripotent stem cells are considered to be distinct from the pluripotent stem cells which are prepared by dedifferentiated ATSC.

Furthermore, an embodiment of the present invention provides a cytotherapeutic composition including said pluripotent stem cell. That is, since the differentiated stem cells can be differentiated into neurons, myocyte, adipocytes, osteocyte, beta cells, and cartilage cells, the differentiated stem cells can be used for cytotherapeutic medicines for treatment of disease selected from a group consisting of diabetes, connective tissues disease, neuropathic disease, autoimmune disease, muscle damage, osteoporosis and osteoarthritis disease. Furthermore, the differentiated pluripotent stem cells according to an embodiment of the present invention can be used in the preparation of the cytotherapeutic composition.

Furthermore, since DHP-derivative involves in the dedifferentiation of the differentiated cells under hypoxia condition, the composition can be used for preparing the composition and culture medium for dedifferentiation.

Furthermore, an embodiment of the present invention provides a cytotherapeutic method for treatment of an individual, which includes the step of administering the dedifferentiated pluripotent stem cells into the individual.

The cytotherapeutic method may include treatment of disease selected from a group consisting of diabetes, connective tissues disease, neuropathic disease, autoimmune disease, muscle damage, osteoporosis and osteoarthritis disease, but not strictly limited thereto.

### Example 1

### Inducing the Differentiation of ATSC by Treating Hypoxia/DHP-Derivative

### <1-1> Extracting DHP-Derivative

The purification process of 4-(3,4-Dihydroxy-phenyl)-derivative (DHP-derivative) will be explained. 4-(3,4-Dihydroxy-phenyl) (DHP) derivative purified from phellinus linteus, a medicinal fungus known as “Sang-hwang” in Korea for cell reprogramming. 1 kg of Phellinus linteus was grinded or cut into small pieces, and then soaked in 10 l of ethanol for 1 week, extracted and filtered to thus obtain 45 g in dry weight. Dried Phellinus linteus extract was mixed with the same amount of hexane and H₂O and then suspended. From the extract, H₂O layer was isolated, and then hexane extract was subjected to repeated silica gel (0.063-0.200 mm, Merck) column chromatography, using a hexane-ethyl acetate gradient as the eluting solvent to obtain ethyl acetate extract. Silica gel column chromatography was conducted with respect to 5.6 g of ethyl acetate extract to obtain 0.4 g of DHP-derivative fraction which has free radical removal activities. The removal activities of the free radical were measured by a known method (Korea Patent No. 10-0609486) and such obtained DHP-derivative was diluted to 10 ng/ml on a culture medium to use in an experiment.

### <1-2> Isolating and Culturing Adipose Tissue-Derived Stem Cells

Adipose tissues were collected from patients of avascular necrosis or fracture in their 60s and 70s. To obtain adipose tissues, adipose tissues were cut from a hip joint of patients by surgical procedure, put in α-MEM medium, went through centrifugal filtration, and then kept in polypropylene at −70° C. Collection of adipose tissues was approved by the patients in prior in a written form. Institutional Review Board also approved of the collecting biochemical materials and information of patients for the purpose of research.

A known method was used to isolate adipose tissue-derived stem cells; adipose tissue stromal cells (ATSC) from patent-derived non-processed adipose tissues [Kang S K et al., 2003, Exp Neurol., 183(2): 355-66; Kang S K et al., 2004, J Cell Sci., 117: 4289-99]. To be specific, 0.075% of the adipose tissue was decomposed by collagenase IV (Sigma, U.S.A.), and then centrifuged (1200 g, 10 min.) to obtain high density cell precipitate. To dissolve red blood cells mixed in the precipitate, the precipitate was suspended in red blood cell lysis buffer (Biowhittaker, U.S.A.) and then cultured at a room temperature for 10 minutes. The precipitate, from which red blood cells were removed, was cultured overnight in α-MEM medium (GIBCO BRL, U.S.A.) [which contains 10% FBS (GIBCO BRL, U.S.A.) and 1% of antibiotics (GIBCO BRL. U.S.A.)] at 37° C. with 5% of carbon dioxide to obtain stem cells.

<1-3> Treating Human ATSC with Hypoxia/DHP-Derivative

Human ATSC was subcultured (P5-P6) in α-MEM medium at 37° C. under 5% of carbon dioxide condition in 5 to 6 generation. To treat the cell line with hypoxia stimulus, a medium which includes 10 ng/ml of DHP-derivative was added to the culture medium on which the cells were cultured, and the sample was cultured for 2 or 6 hours in a chamber (Billups-Rothenberg, U.S.A.) filled with a gas including 1% of oxygen, 5% of carbon dioxide and 94% of nitrogen at atmospheric pressure of 5 to 8 psi. The chamber was fully supplied with moisture, sealed, and additionally cultured at 37° C. As for a negative control group, cells which were treated with DHP-derivative in the same manner as explained above were cultured at a normal oxygen condition (21% of oxygen). After dedifferentiation, dedifferentiated ATSC were replaced with new DHP-derivative (10 ng/ml)-containing culture medium after 48 hours, and the culture medium was replaced once in 4 days.

### Example 2

### Confirming Dedifferentiation of ATSC by Hypoxia/DHP-Derivative Treatment

To confirm the various dedifferentiating shapes of ATSC exposed to hypoxia/DHP-derivative treatment, the inventors conducted experiment.

### <2-1> Confirming Increased Proliferation of ATSC Cells by Hypoxia/DHP-Derivative Treatment

Once differentiating starts, cells are rarely proliferated since the cells are out of cell cycle. To confirm the dedifferentiation of ATSC by hypoxia/DHP-derivative treatment, cell proliferation activity of ATSC of Examples 1 to 3 were measured by flow cytometric analysis and colony forming units (CFU). Also, it was analyzed that such cell proliferation was caused by the increase of telomerase activity.

First, ATSC (experimental group) hypoxia/DHP-derivative, or ATSC (control group) treated with DHP-derivative were cultured to the full extent of 100 mm culture dish, and then upper layer of the medium was collected in 15 ml, of tube (Becton Dickinson, U.S.A.). Cells attached to the culture dish were rinsed with 5 ml PBS two times, and treated with 0.5 ml of 0.15% trypsin to be removed, respectively, and added to the upper layer medium collected earlier as explained above. After that, the tube was centrifuged (200 g, 5 min.) and the cells gravitated. The upper layer solution was removed and the gravitated cells were suspended with 300 ml of PBS, vortexed, and slowly added with 5 ml of cooled 75% ethanol to be fixed. Fixed cells were left at 4° C. for at least 3 hours, and centrifuged again to remove ethanol. After that, cells were added with PBS containing 0.1 mg/ml of RNase (Sigma, U.S.A.) to cell concentration of 1×10⁶ cell/ml, and then added with propidium iodide (1 mg/ml, distilled water, Sigma, U.S.A.) to the final cell concentration of 1×10⁶ cell/ml, and the cells were left at a room temperature for 30 minutes without lights and then stained. At least 10000 stained cells were analyzed per each group by 488 nm of excitation wavelength and 588 nm of emission wavelength of FACScan argon laser cytometer (Becton Dickinson, U.S.A.). Cell percentage at cell cycle of G0/G1, S and G2/M was analyzed by modifit software (Veriety software house, U.S.A). FIG. 1a shows the result of the above analysis.

Also, cells of experimental group or control group were injected into 60 mm of culture medium, and cultured in a DHP-derivative (10 ng/ml)-containing medium for 10 to 15 days. The upper layer was remove from the medium, and the medium was rinsed with 5 ml of PBS two times, added with 5 ml of 100% methanol, and left at −20° C. for 20 minutes to fix the cells. After that, the fixed cells were stained with methylene blue (10 mg/ml, ethanol, Sigma, U.S.A.). Under an optical microscope, cells with 50 and more of colonies were counted. To minimize the error, the above experiments were repeated 3 times and the average values were illustrated in FIG. 1b.

To measure telomerase activities of ATSC and dedifferentiated ATSC, TRP (telomeric repeat amplification protocol) analysis was conducted according to the manual provided by BD Science (U.S.A.). Cells of experimental group or control group were injected into 60 mm culture medium at 1×10⁶ and cultured for 2 days. 0.5 μg of protein extracted from cells of experimental group or control group was cultured with telomerase-specific primer (SEQ ID NO: 1: 5′-AAT CCG TCG AGC AGA GTT-3;) that is, oligonucleotide synthesis and nucleotide (dNTP). If the proteins have telomerase activities, telomerase-specific primer acts as a template, PCR (polymerase chain reaction) occurs, and the oligonucleotide becomes longer. The PCR product was electrophoresed in 12.5% of natured acrylamide gel, and then stained with Syber-Gold dye (Molecular Probes, U.S.A.). The amount of telomerase according to telomerase activities of ATSC and dedifferentiated ATSC was fixed by PCR enzyme-linked immunosorbent assay procedure according to the manual provided by the BD Science. FIG. 1c shows the result.

Referring to FIG. 1a, when ATSC were treated with hypoxia/DHP-derivative for 30 minutes (39.36%) or 6 hours (46.04%), number of M1 cells decreased from the control group (29.36%) and thus the cellular proliferation was more active. FIG. 1b indicates that more colonies formed with the longer treating period of hypoxia/DHP-derivative. FIG. 1c also indicates that the ATSC treated with hypoxia/DHP-derivative for 6 hours exhibited telomerase activity approximately 5 times as high as the control group.

<2-2> Confirming Changes in the Expression of Surface Epitope of ATSC when Treated with DHP-Derivative and Hypoxia Stimulus

When ATSC is dedifferentiated by hypoxia/DHP-derivative treatment, expression of surface epitope and the expression of marker cell of embryonic stem cell were confirmed. Antibodies were purchased from Santa Cruz (U.S.A.) in the following examples unless otherwise specified.

To confirm phenotypic characterization by the method of flow cytometry, cell line of experimental group or control group was suspended with PBS in a concentration of 1×10⁶ cell/ml, and reacted with anti-CD117 antibody, anti-CD44 antibody, anti-CD90 antibody, anti-CD117 antibody, anti-CD113 antibody and anti-CD44 antibody for 20 minutes, and then marked. As for subtype control group, non-specific anti-mouse or anti-rabbit IgG antibody was used instead of primary antibody. The cell marked as antibody was analyzed by FACScan argon laser cytometer. FIG. 2a illustrates the result.

Also, through immunocytochemical analysis, the expression of Sox2[SRY(sex determining region Y)-box 2], SSEA4 (stage specific embryonic antigen 4) and TRA1-80 (tumor rejection antigen 1-80), which are marker proteins of embryonic stem cell, was observed. Cell lines of experimental group or control group were injected into 60 mm of culture medium at a concentration of 1×10⁶ cell/ml, and cultured to a stable state, and fixed in 4% of paraformaldehyde which includes 100 nM of sodium phosphate buffer (pH 7.0) for 15 minutes, and then rinsed in PBS containing 100 mM of glycine for 10 minutes. The cells were treated with IBB (immunofluorescent blocking buffer) which contains 5% of BSA, 10% of FBS, 1×PBS, 0.1% of Triton X-100, and then blocked at a room temperature for 1 hour. The IBB was added with primary antibody (anti-Sox2 antibody, anti-SSEA4 antibody or anti-TRA1-80 antibody) and reacted at 4° C. overnight. After that, the cells were rinsed with PBS/glycine and cultured for one hour in IBB which includes secondary antibody labeled as FITC (fluoroisothiocyanate) or PE (phycoerytherin). The cells were rinsed again with PBS/glycine and stained with a solution which contains DAPI (4,6-diamidino-2-phenylindole, Sigma, U.S.A.) to observe a cell nucleus. The cells were analyzed under a fluorescence microscope and with FACScan argon laser cytometer. FIG. 2b shows the result.

As illustrated in FIG. 2a, it was confirmed that as the time period for hypoxia/DHP-derivative treatment increases, the expression of CD117, CD44, CD90, CD117, CD133 and CD44 increases. FIG. 2b demonstrates that when ATSC was treated with hypoxia/DHP-derivative, the expression of marker protein of embryonic stem cell increases.

<2-3> Confirming Changes in Expression of Gene or Protein when Treated with DHP-Derivative and Hypoxia Stimulus

The inventor confirmed expression of stemness, nerve marker and cell proliferation-related genes or proteins by RT-PCR (real time RT-PCR) or Western blotting when ATSC were dedifferentiated by hypoxia/DHP-derivative treatment.

First, ATSC were cultured in a medium which contains DHP-derivative under the normal oxygen condition (21% of oxygen) or cultured under the hypoxia condition (1% of oxygen) for 30 minutes or 6 hours and the cells were collected. From the cells, the whole RNA was extracted using RNeasy mini-kit (Qiagen, U.S.A.). Distilled water was added to 1 μg of the whole RNA and oligo-dT 1 μl (Invitrogen, U.S.A., 0.5 μg/μl) to 50 μg, and then put into AccuPower RT-premix (Bioneer, Korea) for RT (reverse transcription). The RT was processed at 65° C. for 5 minutes, 4° C. for 5 minutes, 42° C. for 60 minutes, 95° C. for 5 minutes, 4° C. for 5 minutes to synthesize cDNA. The synthesized cDNA was used as a template and real time PCR was conducted in iCycler iQ Real-Time PCR Detection System (Bio-rad, U.S.A.) using Rex1 (Zinc Finger Protein 42; ZFP42), Oct4 (Octamer-4), Runs3 (runt-related transcription factor 3), CDK1 (cyclin dependent kinase 1), CDK2 or β-actin primer pair and PCR Master premix (Promega, U.S.A.). The primer pair of each gene is listed in Table 1. The result of real time PCR is illustrated in FIG. 3a.

TABLE 1

| | Sense primer | | Anti-sense primer | |
|---|---|---|---|---|
| Gene | SEQ ID NO. | Sequence (5′→3′) | SEQ ID NO. | Sequence (5′→3′) |
| Rex1 | SEQ ID NO. 2 | TGAAAGCCCACATCC | SEQ ID NO. 3 | CAAGCTATCCTCC |
| | | TAACG | | TGCTTTG |
| Oct4 | SEQ ID NO. 4 | ACATGTGTAAGCTGC | SEQ ID NO. 5 | GTTGTGCATAGTC |
| | | GGCC | | GCTGCTTG |
| Runs3 | SEQ ID NO. 6 | CTACGGGACATCCTC | SEQ ID NO. 7 | CATCTCTGCCAGC |
| | | TGGCTCC | | AGCGTGCTG |
| CDK1 | SEQ ID NO. 8 | GGCTCTTGGAAATTG | SEQ ID NO. 9 | AGGAACCCCTTCC |
| | | AGCGGA | | TCTTCACT |
| CDK2 | SEQ ID NO. 10 | CTAGCTTTCTGCCATT | SEQ ID NO. 11 | GAAGAGCTGGTCA |
| | | CTCATC | | ATCTCAGAA |
| β-actin | SEQ ID NO. 12 | TTTGAGACCTTCAAC | SEQ ID NO. 13 | AATGTCACGCACG |
| | | ACCCCAGCC | | ATTTCCCGC |

ATSC were cultured in a medium containing DHP-derivative under the normal oxygen condition (21% of oxygen) or cultured under the hypoxia condition (1% of oxygen) for 30 minutes or 6 hours and the cells were collected. The cells were treated with lysis buffer [20 mM HEPES (pH 7.5), 150 mM EGTA, 1% Triton X-100, 10% glycerol and protease I/II (Sigma)] to extract proteins and the expression of Nestin, GFAP (glial fibrillary acidic protein), MAP2ab (microtubule-associated protein2ab), Tuj (neuron-specific class III beta-tubulin), HIF1α (hypoxia-inducible factor 1α, HIF2α, AcetylH3 and AcetylH4 was confirmed by Western blotting. Anti-β-actin antibody was also observed as a quantitative control group along with the expression of β-actin protein. Also, ATSC were cultured in a medium containing DHP-derivative under the normal oxygen condition (21% of oxygen) or cultured under the hypoxia condition (1% of oxygen) for 30 minutes or 6 hours, cells were collected, and the proteins were extracted to confirm the expression of TERT (telomerase reverse transcriptase), Oct4, Sox2[SRY(sex determining region Y)-box2], Nanog and Rex protein by Western blotting. Anti-β-actin antibody was also observed as a quantitative control group along with the expression of β-actin protein. The Western blotting was conducted as follows. 10 μl of protein extracted from each cell was spread on 10% of SDS-PAGE gel and then transferred to NC paper. NC paper was then reacted with primary antibody according to each protein, reacted with peroxidase labeled goat anti-rabbit or anti-mouse IgG secondary antibody at a room temperature, and treated with chemiluminescence (Amersham, U.K.) to compare the amount of expression of the respective proteins. The result of Western blotting is illustrated in FIG. 3b.

FIG. 3a indicates that the ATSC treated longer time with hypoxia/DHP-derivative exhibit increased expression of Rex1, Oct4, Runs3, CDK1 and CDK2 proteins. Also, FIG. 3b indicates that the ATSC treated longer time with hypoxia/DHP-derivative exhibit increased expression of Nestin, HIF1α and AcetylH3 increases, but decreased GFAP, MAP2as, Tju, HIF2a and AcetylH4. When ATSC were treated with hypoxia/DHP-derivative, the expression of TERT, Oct4, Sox2, Hanog and Rex protein increases compared to the case without the above treatment.

### Example 3

### Confirming Dedifferentiation of ATSC Treated with DHP-Derivative and Hypoxia Stimulus by Karyotyping Analysis

Karyotyping analysis can only be conducted in proliferating cells. Since cancer cells are abnormally proliferated due to abnormal expression of genes such as absence or addition of chromosomes, the possibility is very high that there is no normal chromosomes in karyotyping analysis. Stem cells are active in proliferation like cancer cells but proliferate according to the cycle of normal cells, and therefore, have normal chromosomes. The karyotyping analysis was conducted to prove that ATSC treated with hypoxia/DHP-derivative were not turned into cancer cells but dedifferentiated. FIG. 4 illustrates the analyzing result of chromosomes separated from experimental or control group.

As a result, FIG. 4 indicates that both ATSC and dedifferentiated ATSC had normal chromosomes.

### Example 4

### Confirmation of Dedifferentiation

From the above result, it was confirmed that ATSC can be dedifferentiated by the treatment of hypoxia/DHP-derivative. To confirm if completely differentiated cells can be dedifferentiated with hypoxia/DHP-derivative treatment, the analysis of chromosomes and gene expression was conducted with osteocyte completely differentiated from ATSC and adipose tissue derived from ATSC. By a known method, ATSC were differentiated into osteocyte and adipose tissue for use in the experiment [Kim J H et al., 2008, STEM CELLS, 26(10):2724-34].

Osteocyte and adipose tissue which had been completely differentiated, and osteocyte and adipose tissue which had been treated with hypoxia/DHP-derivative were H&E stained, and the changes in cell morphology were observed under a microscope. FIG. 5a shows the result.

The cell proliferation of completely differentiated osteocyte, osteocyte treated with hypoxia/DHP-derivative, and cells of control group were confirmed by using the same method of <Example 2-1> and the result is illustrated in graphical representation of FIG. 5b.

Real time PCR were conducted in the same manner as explained above with reference to Examples 2 and 3 to confirm the expressions of Oct4, Nanog, CDK1, CDK2, Rex1, Runx3 and β-actin in the completely differentiated osteocyte and adipose tissue, osteocyte and adipose tissue treated with hypoxia/DHP-derivative, and cells of control group, respectively. Western blotting same as that of Examples 2 and 3 was also conducted to confirm the expressions of p53, c-myc(myelocytomatosis oncogene) and p21 protein. Herein, primer of Table 2 was used as the real time PCR. Also, real time PCR same as that of Examples 2 and 3 was conducted to confirm the expression of osteonectin, RXR (retinoid-X-receptor) and osteopontin of the completely differentiated adipose tissue, adipose tissue treated with hypoxia/DHP-derivative and cells of control group, respectively. Western blotting same as that of Examples 2 and 3 was also conducted using anti-HIF1a antibody to confirm expression of HIF1a protein. The result is illustrated in FIG. 5c.

TABLE 2

| | Sense primer | | Anti-sense primer | |
|---|---|---|---|---|
| Gene | SEQ ID NO. | Sequence (5′→3′) | SEQ ID NO. | Sequence (5′→3′) |
| Nanog | SEQ ID NO. 14 | TCTGTTTCTTGACTG | SEQ ID NO. 15 | GCTGAGATGCCTCA |
| | | GGACCTTGTC | | CACGGAG |

FIG. 5a demonstrates that osteocyte and adipose tissue were dedifferentiated and cytomorphologically changed. FIG. 5b indicates that dedifferentiated osteocyte and adipose tissue had similar cell proliferation as ATSC which were not yet differentiated. Also, FIG. 5c indicates that dedifferentiated osteocyte and adipose tissue exhibit increased expressions of Oct4, Nanog, CDK1, CDK2, Rex1 and Runx3 gene, c-Myc and p21 but decreased expression of p53. Also, HIF1 α, which is the hypoxia induced transcription factor, was not expressed in ATSC but expressed in dedifferentiated adipose tissue and osteocyte.

### Example 5

### Dedifferentiation of ATSC Treated with DHP-Derivative and Hypoxia

<5-1> Functional Classification of Gene Expressed ATSC which were Treated with DHP-Derivative and Hypoxia Stimulus

For samples of gene array analysis, the whole RNA was extracted from ATSC, dedifferentiated ATSC and human embryonic stem cell (hESC) by using RNeasy mini-kit (Qiagen, U.S.A.) and the microarray analysis was conducted according to the manual provided by Qiagen. For comparison, cRNA segment (15 mg) was hybridized in HG-U954 array (Affymetrix. U.S.A.) at 45° C. for 16 hours. After the hybridization, the probe array was scanned on Agilent with 3 mm resolution by using Genechip system which was prepared for Affiymetrix. Affymetrix microarray uses 4 sheets to scan and analyze the relative expressing amount of genes from the average differences of fluorescence strength. The analysis result of microarray was translated into technical terms such as Unigene or Genebank and then stored in Excel data sheet (Microsoft Corp., U.S.A.). Dedifferentiated ATSC or expressing amount of hESC gene/expressing amount of ATSC gene was calculated into the changed amount of gene expression, and then gene with the amount of expression change greater than 2 times were selected as gene of meaningful changes.

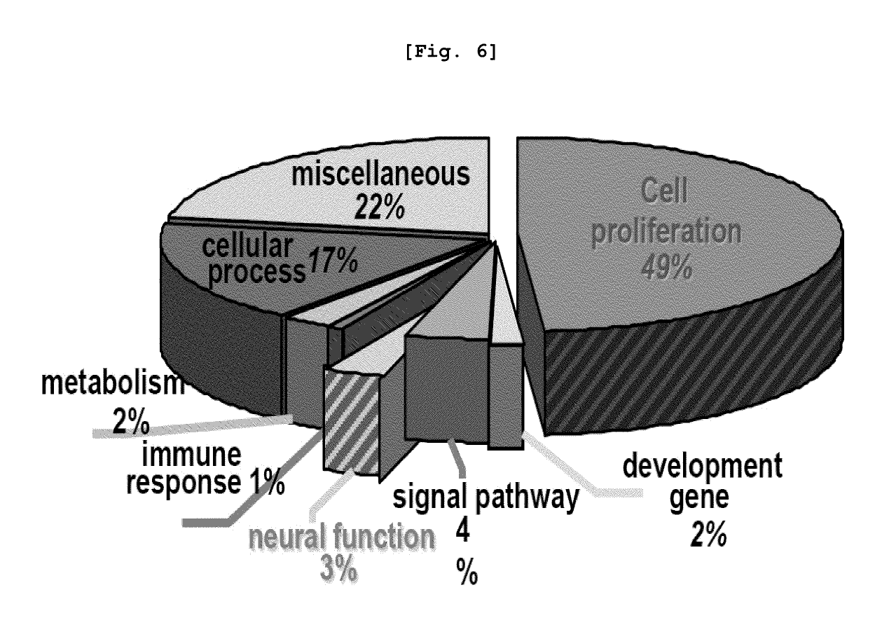

FIG. 6 shows the graphical representation of analysis of functions of the genes of the selected genes in which dedifferentiated ATSC had expression amount two times as high as ATSC or greater than that. Table 3 also lists the result of microarray of histon, DNA methylation and genes of general transcription factors.

TABLE 3

| | Chromosome | Fold |
|---|---|---|
| Gene Title | Location | Change |
| histone 1, H3h | 6p22-p21.3 | 3.61 |
| histone 1, H3a | 6p21.3 | 2.9 |
| histone 1, H3i | 6p22-p21.3 | 6.01 |
| histone 1, H3e | 6p21.3 | 3.2 |
| histone 1, H4c | 6p21.3 | 2.12 |
| histone 1, H4f | 6p21.3 | 11.71 |
| histone 1, H4d | 6p21.3 | 8.13 |
| histone 1, H4h | 6p21.3 | 4.76 |
| GATA binding protein 3 | 10p15 | 3.15 |
| methylthioadenosine phosphorylase | 9p21 | 3.99 |
| serine hydroxymethyltransferase 1 | 17p11.2 | 2.48 |
| forkhead box P1 | 3p14.1 | 0.09 |
| methyl-CpG binding domain protein 2 | 18q21 | 0.41 |
| forkhead box O3A | 6q21 | 0.23 |
| methyl-CpG binding domain protein 2 | 18q21 | 0.41 |
| histone 1, H2bd | 6p21.3 | 0.15 |
| histone 1, H4e | 6p21.3 | 0.15 |
| histone 2, H4a | 1q21 | 0.15 |
| histone 1, H4a | 6p21.3 | 0.18 |
| histone 1, H4k; histone 1, H4j | 6p22-p21.3 | 0.33 |
| histone 1, H4b | 6p21.3 | 0.38 |
| histone 1, H2bn | 6p22-p21.3 | 0.18 |
| histone 1, H2ac | 6p21.3 | 0.39 |
| histone 2, H2aa3 | 1q21.2 | 0.49 |
| CCAAT/enhancer binding protein | 8p11.2-p11.1 | 0.35 |
| GATA binding protein 6 | 18q11.1-q11.2 | 0.33 |

FIG. 6 indicates that, in the dedifferentiated ATSC, 49% of cell proliferation-related gene, 17% of cell processing related genes, 4% of signal transduction gene, 3% of neurility gene, 2% of each metabolism related gene and development related gene, 1% of immune related gene, and 22% of other gene were increased. Also, as listed in Table 3 above, genes of general transcription factors were increased approximately 2 to 11 times, but the expression of histone and DNA methyl related genes are all decreased.

<5-2> Comparison of ATSC or Dedifferentiated ATSC and hESC Gene

The differences between gene expressed from ATSC and hESC, and gene expressed from dedifferentiated ATSC and hESC were analyzed from the microarray result of the <Examples 5-1> and listed in FIG. 7a. Among genes from dedifferentiated ATSC and hESC which were not expressed in ASTC, genes of embryonic period (Utf1, Dapp5, FGF4 and Eras) were analyzed for the expressing amount compared to other genes by RT-PCR in the same manner as explained above with reference to Examples 2 and 3, and the result is illustrated in FIG. 7b. Primer used in the RT-PCT is listed in Table 4.

TABLE 4

| | Sense primer | | Anti-sense primer | |
|---|---|---|---|---|
| Genes | SEQ ID NO. | Sequence (5′→3′) | SEQ ID NO. | Sequence (5′→3′) |
| Utf1 | SEQ ID NO. 16 | CCGTCGCTGAACA | SEQ ID NO. 17 | CGCGCTGCCCAG |
| | | ACGCCCTGCTG | | AATGAAGCCCAC |
| Dapp5 | SEQ ID NO. 18 | TGAAAGATCCAGA | SEQ ID NO. 19 | ACTGGTTCACTT |
| | | GGTGTTC | | CATCCAAG |
| FGF4 | SEQ ID NO. 20 | CTA CAA CGC CTA | SEQ ID NO. 21 | GTT GCA CCA |
| | | CGA GTC CT | | GAA AAG TCA GA |
| ERas | SEQ ID NO. 22 | GCTGTCTGTGATG | SEQ ID NO. 23 | TCTCCAGCAGTG |
| | | GTGTGCT | | GTCACAAG |

As shown in FIG. 7a, compared to the number of genes simultaneously expressed in ATSC and hESC, the number of genes simultaneously expressed in dedifferentiated ATSC and hESC are approximately 5 times greater. Also, FIG. 7b demonstrates that the expression of genes of embryonic period increased in dedifferentiated ATSC.

### Example 6

### Comparison of Genes of ATSC and Dedifferentiated ATSC by Promoter Regions Methylation Analysis

Not all the genes within cells are expressive. The expression of genes is controlled by the function of the cells, environment, or period. Part of genes can be suppressed from expression when promoter is methylated. Methylation of gene is one of the methods to turn on/off the gene switch. One of the significant differences between differentiated cells and non-differentiated cells is the different pattern of DNA methylation. Accordingly, the inventors observed the different pattern of methylation between cells of experimental group and control group to confirm the relationship between the change of expressing pattern of the genes and DNA methylation. Bisulite of DNA of genes of each cell line was analyzed regarding the transformation and sequence to analyze the promoter methylation pattern of genes of embryonic period (REX1, OCT4 and SOX2).

To be specific, extracting phenol/chloroform/isoamyl alcohol, chloroform from experimental group and control group and rinsing with ethanol were carried out in sequence to obtain genomic DNA. The DNA was dissolved in water and transformed into bisulite using EZ DNA MethylationGold kit (Zymo Research, U.S.A.) according to the instructions. Briefly put, non-methylated cytosine of DNA was transformed into uracil by heat using CT converter which is specifically designed. After that, the NDA was desulphonated, rinsed and dissolved. The bisulite-transformed DNA was used as DNA to conduct PCR in MyGenie 96 Gradient Thermal Block (Bioneer, U.S.A.). Primer (MethPrimer, Table 5) used in the PCR was designed with reference to the website at “//www.urogene.org/methprimer”. The PCR product was cloned in bacteria (DH5α) using pGEM T-Easy Vector System I (Promega, U.S.A.) and sequenced as anti-sense primer (SEQ NO: 24: 5′-AGCGGATAACAATTTCACACAGGA-3′) using ABI 3730XL capillary DNA sequencer (Applied Biosystems, U.S.A.). The sequencing result is illustrated in FIG. 8. One white hexagon in a row represents one nucleotide, and one black hexagon represents one CpG methylation.

FIG. 8 demonstrates that REX1 has decreased methylation in promoters 1 and 2 in dedifferentiated ATSC than ATSC, OCT4 has decreased methylation in promoter 1 and 3 in dedifferentiated ATSC than ATSC, and SOX2 has no changes in methylation in promoter.

TABLE 5

| The | | | | | applied | |
|---|---|---|---|---|---|---|
| name | | | | | scope of | Annealing |
| of | Primer | Sequence (5′->3′) | | | sequence | temperature |
| gene | pair | Normal direction[F]/reverse direction[R] | | | to TSS | (° C.) |
| OCT4 | OCT4_1 | SEQ NO. 25 | F | TTTTTAGTTTTTTTTAGGT | −2995 to | 51 |
| | | | | TTAA | −2723 | |
| | | SEQ NO. 26 | R | TAAACAAAAAACCCATTC | | |
| | | | | CC | | |
| | OCT4_2 | SEQ NO. 27 | F | TTAGGAAAATGGGTAGTA | −2609 to | 58 |
| | | | | GGGATTT | −2417 | |
| | | SEQ NO. 28 | R | TACCCAAAAAACAAATAA | | |
| | | | | ATTATAAAACCT | | |
| | OCT4_3 | SEQ NO. 29 | F | ATTTGTTTTTTGGGTAGTT | −2344 to | 58 |
| | | | | AAAGGT | −2126 | |
| | | SEQ NO. 30 | R | CCAACTATCTTCATCTTAA | | |
| | | | | TAACATCC | | |
| | OCT4_4 | SEQ NO. 31 | F | GGATGTTATTAAGATGAA | −2136 to | 58 |
| | | | | GATAGTTGG | −1721 | |
| | | SEQ NO. 32 | R | CCTAAACTCCCCTTCAAA | | |
| | | | | ATCTATT | | |
| | OCT4_5 | SEQ NO. 33 | F | GAAGGGGAAGTAGGGATT | −1014 to | 58 |
| | | | | AATTTT | −720 | |
| | | SEQ NO. 34 | R | CAACAACCATAAACACAA | | |
| | | | | TAACCAA | | |
| | OCT4_6 | SEQ NO. 35 | F | TAGTTGGGATGTGTAGAG | −567 to | 58 |
| | | | | TTTGAGA | −309 | |
| | | SEQ NO. 36 | R | TAAACCAAAACAATCCTT | | |
| | | | | CTACTCC | | |
| | OCT4_7 | SEQ NO. 37 | F1 | ATAAAGTGAGATTTTGTTT | −202 to | 50 |
| | | | | TAAAAA | +231 | |
| | | SEQ NO. 38 | R1 | AACATAAAAAAATCCCCC | | |
| | | | | ACAC | | |
| | | SEQ NO. 39 | F2 | GGGATTTGTATTGAGGTTT | | 56 |
| | | | | TGG | | |
| | | SEQ NO. 40 | R2 | CCCACACCTCAAAACCTA | | |
| | | | | AC | | |
| NANOG | NANOG_1 | SEQ NO. 41 | F | AGAGATAGGAGGGTAAGT | −1503 to | 58 |
| | | | | TTTTTTT | −1254 | |
| | | SEQ NO. 42 | R | ACTCCCACACAAACTAAC | | |
| | | | | TTTTATTC | | |
| | NANOG_2 | SEQ NO. 43 | F | GAGTTAAAGAGTTTTGTTT | −1203 to | 52 |
| | | | | TTAAAAATTAT | −911 | |
| | | SEQ NO. 44 | R | TCCCAAATCTAATAATTTA | | |
| | | | | TCATATCTTTC | | |
| | NANOG_3 | SEQ NO. 45 | F | TTAATTTATTGGGATTATA | −334 to | 58 |
| | | | | GGGGTG | −163 | |
| | | SEQ NO. 46 | R | AACAACAAAACCTAAAAA | | |
| | | | | CAAACC | | |
| SOX2 | SOX2_1 | SEQ NO. 47 | F | GTAGGTTGGTTTTGGGAG | −268 to | 55 |
| | | | | TTTTT | −52 | |
| | | SEQ NO. 48 | R | AATTAATAAACAACCATC | | |
| | | | | CATATAAC | | |
| | SOX2_2 | SEQ NO. 49 | F | TGTTTTTTTAAGATTAGGA | +80 to | 53 |
| | | | | TTGAGAGAA | +253 | |
| | | SEQ NO. 50 | R | AAAACAAACTAAAATCAA | | |
| | | | | AATCAAA | | |
| | SOX2_3 | SEQ NO. 51 | F | ACAAACTAACTCTAAAAA | +427 to | 43 |
| | | | | CC | +616 | |
| | | SEQ NO. 52 | R | GGTTGTTAGGGAATAAAT | | |
| | | | | GG | | |
| | SOX2_4 | SEQ NO. 53 | F | AGATGGTTTAGGAGAATT | +600 to | 55 |
| | | | | TTAAGATGTATA | +965 | |
| | | SEQ NO. 54 | R | AACCCAACTAATCCTACA | | |
| | | | | TCATACTATAAC | | |
| | SOX2_5 | SEQ NO. 55 | F | GGTAGTTATAGTATGATG | +932 to | 55 |
| | | | | TAGGATTAGTTG | +1151 | |
| | | SEQ NO. 56 | R | AACCCATAAAACCAAAAA | | |
| | | | | CCATA | | |
| | SOX2_6 | SEQ NO. 57 | F | GGGATATGATTAGTATGT | +1239 to | 57 |
| | | | | ATTTTTT | +1482 | |
| | | SEQ NO. 58 | R | AATTTTCTCCATACTATTT | | |
| | | | | CTTACTCTCCT | | |
| REX1 | REX1_1 | SEQ NO. 59 | F | AAATATTGGGGGTGTTTG | −868 to | 57 |
| | | | | AAATAAT | −590 | |
| | | SEQ NO. 60 | R | CCCAACTACTCAAAAAAC | | |
| | | | | TAAAACAA | | |
| | REX1_2 | SEQ NO. 61 | F | AAAAGGGTAAATGTGATT | −423 to | 54 |
| | | | | ATATTTA | −68 | |
| | | SEQ NO. 62 | R | CAAACTACAACCACCCAT | | |
| | | | | CAAC | | |
| | REX1_3 | SEQ NO. 63 | F | ATGGGTGGTTGTAGTTTGA | −85 to | 57 |
| | | | | TTAGAT | +279 | |
| | | SEQ NO. 64 | R | TTTCAACATTTAAAACCAA | | |
| | | | | TAACCAA | | |
| | REX1_4 | SEQ NO. 65 | F | TTATTATAAAAGAGTTAG | +2229 to | 54 |
| | | | | GAAGTTTGTATA | +2547 | |
| | | SEQ NO. 66 | R | ATTACCCAAACTAAAATA | | |
| | | | | CAACAAC | | |
| | REX1_5 | SEQ NO. 67 | F | TTTGGAGGAATATTTGGTA | +7497 to | 51 |
| | | | | TTGATT | +7889 | |
| | | SEQ NO. 68 | R | CCTATTACAACCTTAAAAA | | |
| | | | | AAACACAC | | |
| TERT | TERT | SEQ NO. 69 | F | CTACCCCTTCACCTTCCAA | −151 to | 57 |
| | | | | | +164 | |
| | | SEQ NO. 70 | R | GTTAGTTTTGGGGTTTTAG | | |
| | | | | G | | |

### Example 7

### Confirming Growth Promotion by Growth-Related Signal Pathway in Dedifferentiated ATSC

### <7-1> Confirming JAK/STAT3 and MEK Signal Transmission Activity in Dedifferentiated ATSC

The inventors conducted following experiment to confirm the expression pattern of growth-related signal protein and Rex-1 in ATSC treated with hypoxia/DHP-derivative. To be specific, the expression of P-Jak2 (Phosphorylated-Janus kinase 2) and P-Stat3 (Phosphorylated-signal transducer and activator of transcription3) was observed by Western blotting using anti-P-Jak2 antibody and anti-P-Stat3 antibody in the same manner explained above in Examples 2 and 3 in cells of control group not treated with AG490 (Jak2 inhibitor, Sigma) and in cells of experimental group not treated with AG490 or treated with 20 μM or 30 μM. The expression of p-STAT3 and STAT3 was observed by Western blotting using anti-p-STAT3 antibody and anti-Stat3 antibody in the same manner of the Examples 2 and 3 in a control group not treated with AG490 and experimental group treated with hypoxia/DHP-derivative for 6 hours and then treated or not treated with AG490. The expression of p-MEK (Phosphorylated-Mitogen-activated Protein/ERK Kinase), MEK, p-ERK (Phosphorylated-Extracellular signal-regulated kinases), ERK, C-myc, p-STAT3 and STAT3 proteins was observed by Western blotting in the same manner of the Examples 2 and 3 in control group not treated with PD98059 (MEX inhibitor, Sigma) and experimental group treated with hypoxia/DHP-derivative for 6 hours and then treated or not treated with PD98059. The expression of p-P38, P38, P53 and P21 protein was observed by Western blotting in the same manner of Examples 2 and 3 in control group not treated with SB203580 (P38MAPK inhibitor, Sigma) and experimental group treated with hypoxia/DHP-derivative for 6 hours and then treated or not treated with SB203580. The antibodies to each protein were purchased from Santa Cruz. The result is illustrated in FIG. 9a.

TABLE 6

| | Sense primer | | Anti-sense primer | |
|---|---|---|---|---|
| Gene | SEQ ID NO. | Sequence (5′→3′) | SEQ ID NO. | Sequence (5′→3′) |
| Utf1 | SEQ ID NO. 93 | CCGTCGCTGAACAAC | SEQ ID NO. 94 | CGCGCTGCCCAGAA |
| | | GCCCTGCTG | | TGAAGCCCAC |
| Dapp5 | SEQ ID NO. 95 | TGAAAGATCCAGAGG | SEQ ID NO. 96 | ACTGGTTCACTTCAT |
| | | TGTTC | | CCAAG |
| FGF4 | SEQ ID NO. 97 | CTACAACGCCTACGA | SEQ ID NO. 98 | GTTGCACCAGAAAA |
| | | GTCCT | | GTCAGA |
| ERas | SEQ ID NO. 99 | GCTGTCTGTGATGGT | SEQ ID NO. 100 | TCTCCAGCAGTGGT |
| | | GTGCT | | CACAAG |

The expression of Rex1, CDk1 and Runx3 genes were observed by real time PCR in the same manner of the Examples 2 and 3 in cells of control group not treated with AG490 and cells of experimental group not treated with AG490 or treated with 20 μM or 30 μM. As for a quantitative control group, the expression of GAPDH (glyceraldehyde-3-phosphate dehydrogenase) was also observed.

Expressions of REX1, Oct-4, SOX2, CDk1 and CDk2 were observed by real time PCR in the same manner of Examples 2 and 3 in each of the following cases. ATSC were treated with hypoxia/DPH-derivative for 6 hours and then divided into cases including: i) sample treated with nothing; ii) sample treated with SB203580 only; iii) sample treated with PD98059 only; and iv) sample treated with SB203580 and PD98059. The used primers are listed in Table 6. As for a quantitative control group, the expression of GAPDH was also confirmed. The result is illustrated in FIG. 9b.

FIG. 9a indicates that dedifferentiated ATSC treated with higher concentration of AG490 had decreased expression of P-Jak2 and P-Stat3. Also, compared to the sample not treated with PD98059, dedifferentiated ATSC treated with PD98059 had increased phosphorylation of MEK, decreased expression of C-myc, and increased phosphorylation. Also, compared to the sample not treated with SB203580, the dedifferentiated ATSC treated with SB203580 had decreased phosphorylation of P38, and increased expression of P53 and P21. FIG. 9b also indicates that the dedifferentiated ATSC treated with higher concentration of AG490 had decreased expression of Rex′1, CDK1, CDK2, Nanog and Runx3. When dedifferentiated ATSC were treated with SB203580 or PD98059, the expression of REX1, SOX2, CDk1 and CDk2 was decreased but the expression of Oct-4 was not changed.

### <7-2> Confirming Activity of Growth-Related Signal Protein in Dedifferentiated ATSC

The inventors conducted following experiment to confirm the actual growth of ATSC treated with hypoxia/DHP-derivative by the activity of Rex-1 and growth-related signal protein which was confirmed at Example 7-1. To be specific, cells of control group and cells of experimental group treated with HPD98059, SB203580 or AG490, or not treated were prepared, and measured for colony formation. The result is illustrated in FIG. 10.

FIG. 10 indicates that compared to the non-treated experimental group, dedifferentiated ATSC treated with HPD98059, SB203580 or AG490 had decreased cell proliferation which is similar to the cells of control group.

### Example 8

### Confirming Stemness Gene Expression in Dedifferentiated ATSC by Transcription Factor

### <8-1> Confirming Reduced Expression of Cellular Proliferation-Related Genes and Stemness Gene Due to Suppressed Expression of Transcription Factor in Dedifferentiated ATSC

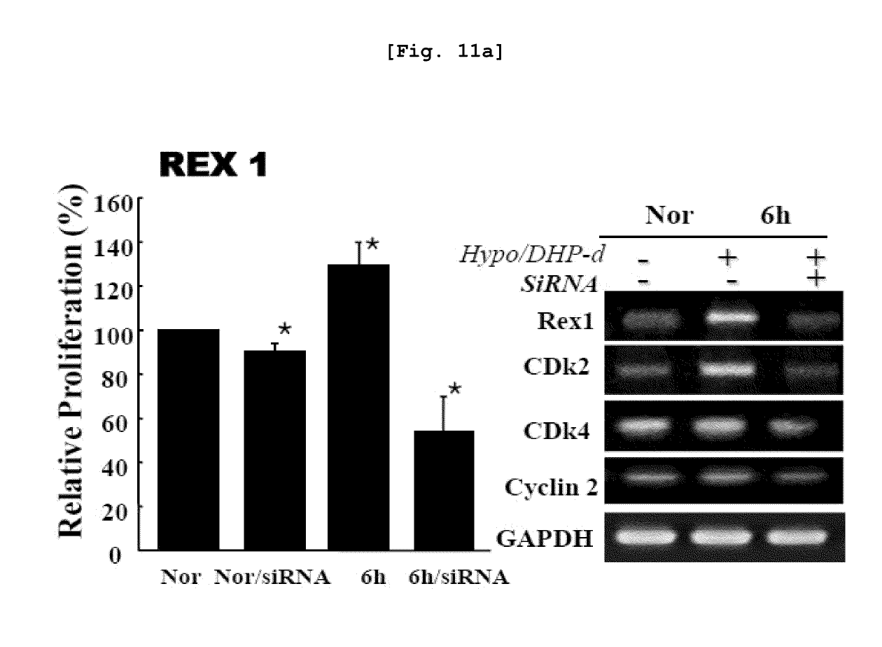

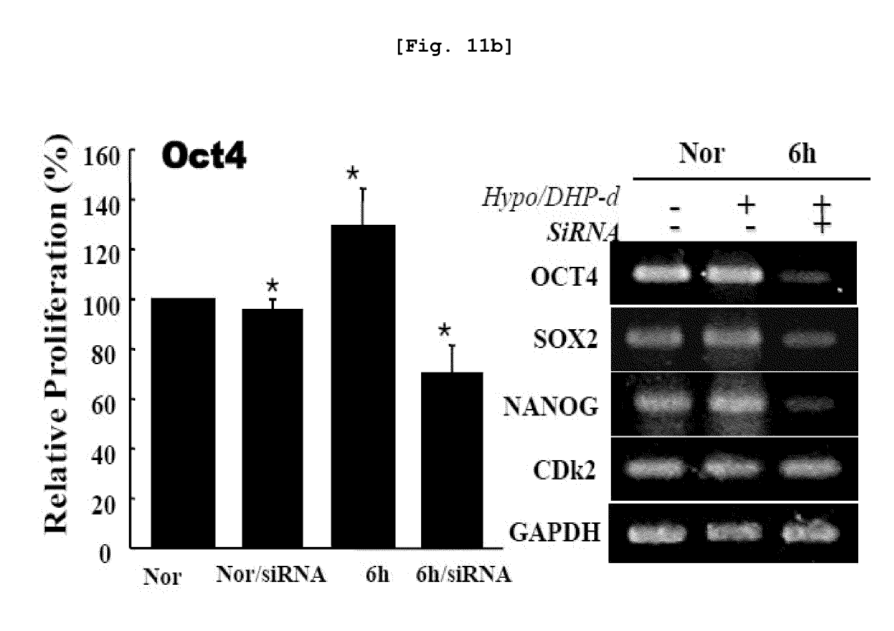

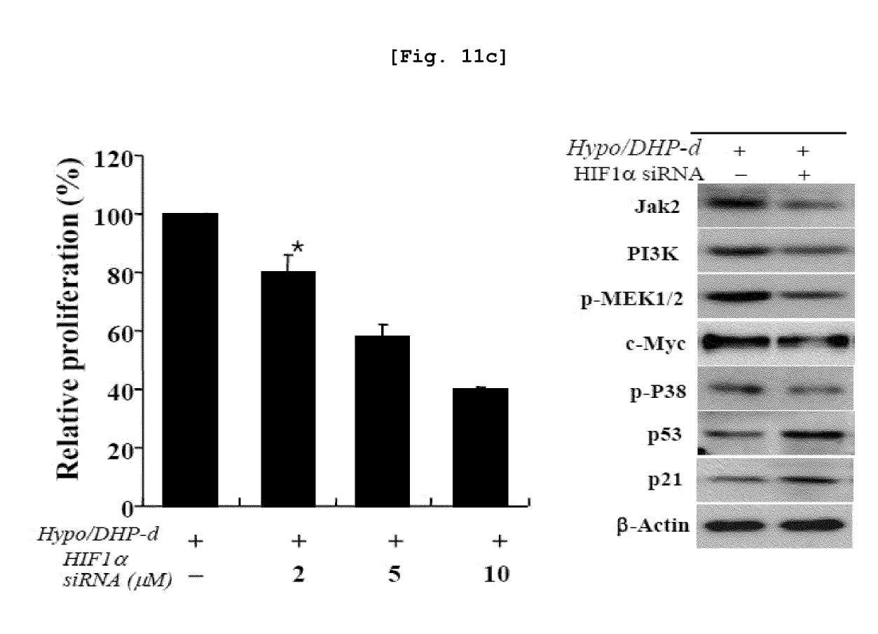

In order to characterize the role of REX1 (transcription factor over-expressed in embryonic stem cell), Oct4 (transcription factor which is over-expressed in stem cell and which provides pluripotency of stem cell, and HIF1 α (transcription factor of which expression is induced in hypoxia condition and which prevents aging of cells) in dedifferentiated ATSC, the inventors treated cells with siRNA with respect to the respective genes and then observed expression of stemness gene and cellular proliferation-related genes and changes in cellular proliferation. First, experimental and control cell groups 3×10⁵ were seeded in 60 mm culture dish, and cultured in a α-MEM medium containing 10 ng/ml DHP-derivative in 37° C., 5% carbon dioxide incubator. 2 μg of REX1 siRNA (Dharmacon RNA Technology, U.S.A) was transfected into the respective cultured cells using Lipofectamine RNAi max (invitrogen, U.S.A). Formation of colonies was measured from the cells with the method as explained above in Example 2-1 to observe cellular proliferation, and by conducting RT-PCR in the same manner as that of Example 2-3, expression of Rex1, CDk2, CDk4 and Cyclin2 (sense primer: SEQ. ID No. 101, 5′-GAG AAG CTG TCC CTG ATC CGC AAG C-3′, antisense primer: SEQ. ID No. 102, 5′-AGA CTT GGA GCC GTT GTG CTG CTC-3′). The expression of GAPDH was also observed as quantitative control group and the result is listed in FIG. 11c.

2 μg of Oct4 siRNA (Dharmacon RNA Technology, U.S.A) was transfected into experimental and control cell groups in the same manner as explained above, and formation of colonies was measured in the same manner as explained in Example 2-1 to observe cellular proliferation, and RT-PCR was conducted in the same manner as explained in Example 2-3 to observe expression of OCT4, SOX2, NANOG and CDk2 genes. GAPDH expression was also observed as a quantitative control group and the result is listed in FIG. 11b.

Additionally, either transfection of siRNA into dedifferentiated ATSC was omitted, or 2 μg, 5 μg or 10 μg of HIF1α siRNA (Dharmacon RNA Technology, U.S.A) was transfected in the manner as explained above. Cellular proliferation was observed by measuring formation of colonies in the cells in the same manner as explained above in Example 2-1, and expression of Jak2, PI3K, p-MEK1/2, c-Myc, p-P-38, P53 and P21 proteins was observed by conducting Western blotting in the same manner as explained above in Example 2-3. Antibodies with respect to the respective proteins were purchased from Santa Cruz Biotechnology Inc. and used. Expression of β-actin was also observed as for a quantitative control group and the result is listed in FIG. 11c.

As a result, FIG. 11a demonstrates that the ATSC treated with REX siRNA had reduced cellular proliferation rate by half or more compared to the non-treated example, and also exhibited reduced expression of Rex1, CDk2, CDk4 and Cyclin2 genes. FIG. 11b indicates that the dedifferentiated ATSC treated with Oct4 siRNA had reduced cellular proliferation rate by half or more compared to the non-treated example, and exhibited reduced expression of OCT4, SOX2 and NANOG but no changes in the expression of CDk2. Additionally, FIG. 11c indicates that the example treated with higher concentration of HIF1 α siRNA had less cellular proliferation, and also had less expression of Jak2, PI3K, p-MEK1/2, c-Myc and p-P-38, but more expression of P53 and P21.

### <8-2> Signal Pathway of Cellular Proliferation-Related Cells of Dedifferentiated ATSC

FIG. 12 shows the flow of signal pathway which regulates expression of intranuclear Rex1, Nanog, p53, p21 and c-myc genes, and expression of sternness genes such as Rex1, Sox2, Oct4 and Klf4 (Kruppel-like factor 4) by HIF1α based on the result of Example 8-1.

### Example 9

### Confirming Increased Cell Migration in Dedifferentiated ATSC

<9-1> Cellular Migration in ATSC in Accordance with Time Period of Treating with DHP-Derivative and Hypoxia Stimulus

In the developmental procedures, cell migration is considered to be critical for normal development. Accordingly, the inventors conducted experiment as explained below to confirm the in vitro migration activity of dedifferentiated ATSC. First, experimental group was cultured in a culture medium containing control cell group and DHP-derivative under hypoxia condition for 30 minutes or 6 hours and then cultured in low serum growth medium. The cultured cells were moved onto 6-transwell plate (8 μm pore size; Costar, U.S.A) coated with laminin (Chemicon, U.S.A). The respective cells were divided on an upper layer chamber, and cultured under 37° C., 5% carbon dioxide condition for 48 hours, during which the cells were left to migrate from the upper layer chamber to the lower layer chamber. The remaining, non-migrated cells were removed from the surface of the upper layer chamber and the migrated cells in the lower layer chamber were stained with Harris hematoxylin (Sigma Aldrich, U.S.A). Cells were counted from 20-selected areas for the respective cells.

Additionally, RT-PCR was conducted in the same manner as explained above in Example 2-3 with respect to the cells, so that expression of cell-migration related genes such as MMP2 (editMatrix metallopeptidase 2), MMP9, SDF1 (stromal cell-derived factor-1), PDGFRa (Platelet-derived growth factor receptor, alpha polypeptide) and VEGF (Vascular endothelial growth factor) was observed. GAPDH expression was also observed as for a quantitative control group and primer used in the experiment is listed in Table 7 below. The result is listed in FIG. 13.

As a result, FIG. 13 indicates cell migration increases in ATSC as the time for treating with hypoxia/DHP-derivative increases, and expression of cellular proliferation related genes also increases.

TABLE 7

| | Sense primer | | Antisense primer | |
|---|---|---|---|---|
| | SEQ. ID. | | SEQ. ID. | |
| Gene | No. | Base sequence (5′→3′) | No. | Base sequence (5′→3′) |
| MMP2 | SEQ. ID. | ACGACCGCGACAA | SEQ. ID. | CTGCAAAGAACACA |
| | No. 103 | GAACTAT | No. 104 | GCCTTCTC |
| MMP9 | SEQ. ID. | CCCGTCCTGCTTTG | SEQ. ID. | ATCCAAGTTTATTA |
| | No. 105 | CAGT | No. 106 | GAAACACTCCA |
| SDF1 | SEQ. ID. | TTGCCAGCACAAA | SEQ. ID. | CTCCAAAGCAAACC |
| | No. 107 | GACACTCC | No. 108 | GAATACAG |
| PDGFRa | SEQ. ID. | ATCAATCAGCCCA | SEQ. ID. | TTCACGGGCAGAAA |
| | No. 109 | GATGGAC | No. 110 | GGTACT |
| VEGF | SEQ. ID. | ACATCTTCCAGGAG | SEQ. ID. | GCATTCACATTTGTT |
| | No. 111 | TACCCTGATGAG | No. 112 | GTGCTGT |

<9-2> Confirming Expression of Cell Migration-Related Proteins in Dedifferentiated ATSC According to Time Period for Treating with DHP-Derivative and Hypoxia Stimulus Treatment

In order to confirm possible relationship between in vitro migration activity of ATSC treated with hypoxia/DHP-derivative and the time for treating under hypoxia stimulus, the inventors have conducted the following experiment. First, by Western blotting, the presence of phosphorylation of cell migration-related proteins of ATSC which were cultured under normal oxygen condition in DHP-derivative containing culture medium, or cultured under hypoxia condition for 1, 2 or 6 hours, was observed. For Western blotting, anti p-ERK antibody, anti ERK antibody, anti p-JUNK (c-Jun N-terminal kinases) antibody, anti JUNK antibody, anti p-P38 antibody and anti p38 antibody were purchased from Santa Cruz Biotechnology Inc. and used. Expression of β-actin was also observed using anti-β-actin antibody as for a quantitative control group. The result is listed in FIG. 14.

As a result, phosphorylation of ERK, JUNK and P38 which are proteins related to cell migration increased in ATSC as the time period for treating with hypoxia/DHP-derivative increased.

<9-3> Confirming Reduction of Cell Migration of Dedifferentiated ATSC by Treatment with Cell Signal Transmission Inhibitor According to Time Period for Treating with DHP-Derivative and Hypoxia Stimulus

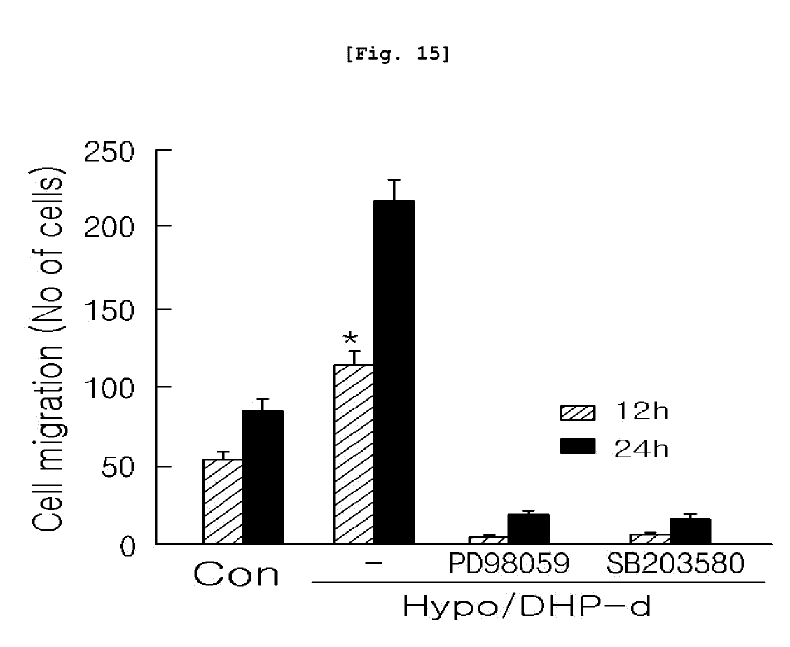

The inventors conducted experiment to confirm the signal pathway that influences the in vitro migration of ATSC which were treated with hypoxia/DHP-derivative. To be specific, in control cell group and experimental cell group treated with PD98059 or SB203580 or treated with none, cell migration was observed in the same manner as explained above in Example 9-1 and the result is expressed in graphical form in FIG. 15.

As a result, FIG. 15 indicates that the cell migration noticeably decreased when the dedifferentiated ATSC were treated with PD98059 or SB203580.

### <9-4> Cellular Signal Pathway Related to Cell Migration by DHP-Derivative and Hypoxia Stimulation Treatment

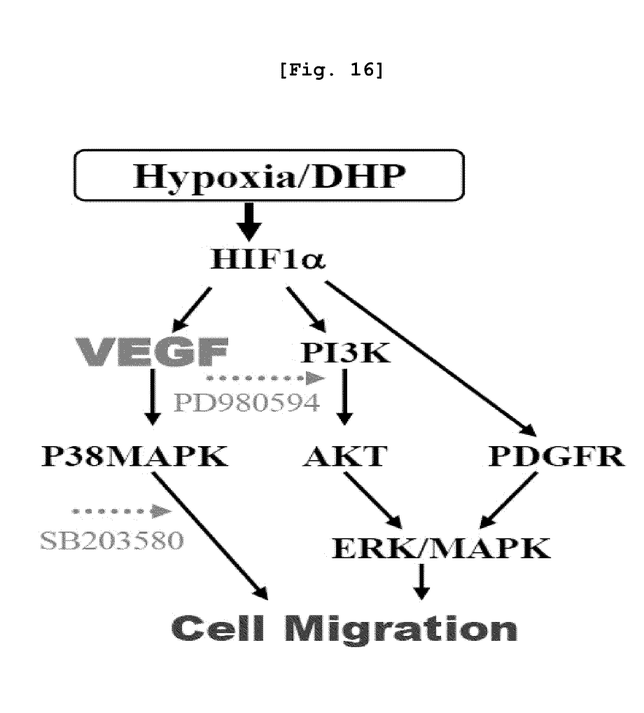

The flow of signal pathway which regulates cell migration of dedifferentiated ATSC is expressed in FIG. 16 based on the result obtained from Examples 9-1 to 9-3.

### Example 10

### Confirming Re-Differentiation Ability of Dedifferentiated ATSC

The inventors conducted experiment to confirm whether or not ATSC, dedifferentiated by hypoxia/DHP-derivative treatment, is able to re-differentiate into various organs in vitro or in vivo.

### <10-1> Confirming In Vitro Re-Differentiation Ability of Dedifferentiated ATSC

The inventors confirmed if the dedifferentiated ATSC by hypoxia/DHP-derivative treatment were re-differentiated into osteocyte or adipocyte in vitro to confirm pluripotency of the dedifferentiated ATSC. To be specific, ATSC cells, cultured either under normal oxygen condition or under hypoxia condition for 30 minutes or 2 hours in a culture medium containing DHP-derivative, were cultured for 2 to 4 weeks in either osteocyte differentiation culture medium [10% FCS, 0.1 μM dexamethasone, 50 μM ascorbate-2-phosphate, 10 mM β-glycerophosphate] or in adipocyte differentiation culture medium [10% FCS, 0.5 mM isobutyl-methylxanthine (IBMX), 1 μM dexamethasone, 10 μM insuline, 200 μM indomethacin]. Induction to osteocyte differentiation was confirmed by Alzarin red (Sigma) staining which can measure calcium accumulation of the cells, and induction to adipocyte differentiation was confirmed by Oil Red 0 (Sigma) staining which can indicate adipose accumulation of the cells. The cells were placed in a fixative of 4% formaldehyde/1% calcium solution, rinsed with 70% ethanol and distilled water and examined under a microscope. Cells were counted from 20 selected areas for each of the cells, the counted result was averaged and the ratio of the osteocytes or adipocytes with respect to the total number of cells in the graphical form as showed in FIG. 17. Additionally, RT-PCR was conducted with the primer of Table 8 in the same manner as explained above with respect to Example 2-3 to observe expression of adipogenesis- and osteogenesis-related transcription factors genes such as RXR, Osteopontin, AP (Alkaline phosphatase) and PPAR-γ (peroxisome proliferator-activated receptor gamma). GAPDH expression was also observed as for a quantitative control group.

As a result, FIG. 17 indicate that the ATSC with longer period of hypoxia/DHP-derivative treatment exhibited greater differentiation into adipocyte and osteocyte, and also increased expression of RXR, Osteopontin, AP and PPAR-γ genes.

TABLE 8

| | Sense primer | | Antisense primer | |
|---|---|---|---|---|
| | | Base sequence | | Base sequence |
| Gene | SEQ. ID. No. | (5′→3′) | SEQ. ID. No. | (5′→3′) |
| RXR | SEQ. ID. No. 113 | ACATGGCTTCCTT | SEQ. ID. No. 114 | CAGCTCAGCCTCC |
| | | CACCAAG | | AGGATCC |
| Osteopontin | SEQ. ID. No. 115 | GCTCTAGAATGAG | SEQ. ID. No. 116 | GTCAATGGAGTCC |
| | | AATTGCACTG | | TGGCTGT |
| AP | SEQ. ID. No. 117 | TGAAATATGCCCT | SEQ. ID. No. 118 | TCACGTTGTTCCT |
| | | GGAGC | | GTTTAG |
| PPAR-γ | SEQ. ID. No. 119 | AGGCGAGGGCGA | SEQ. ID. No. 120 | ACCAGGAATGCTT |
| | | TCTTGACA | | TTGGCATACTCT |

### <10-2> Confirming In Vivo Re-Differentiation Ability of Re-Differentiated ATSC

The inventors confirmed if dedifferentiated ATSC with hypoxia/DHP-derivative treatment were re-differentiated into osteocyte, cartilage cell or myocyte in animal models to confirm pluripotency of the dedifferentiated ATSC. To be specific, a mixture of ATSC or dedifferentiated ATSC cells (2×10⁶ pieces) and Matrigel (BD Biosciences, U.S.A) was implanted into tail vein of 8-week-old SCID/NOD mouse (Orientbio Inc., Korea) using 25-gage needle (Nunc, U.S.A). After 6 weeks, 0.9% normal saline solution was refluxed through aorta ascendens of the mouse, and then 0.1 M phosphate buffer containing 4% paraformaldehyde was refluxed. After that, organs (including bones, muscles and cartilage) were removed and placed in a fixative at 4° C. The fixed organs were sectioned into 40 μm thickness using microtome. The sections were reacted in 3% peroxide hydrogen dissolved in 0.1 M PB (pH 7.4) for 30 minutes and rinsed with PB. By Alzarin Red, Masson (Sigma) and Van Gieson (Sigma) staining, osteocyte, myocyte, and cartilage cells were observed in the sections. The result of observation is shown in FIG. 18.

As a result, FIG. 18 indicates that the mouse implanted with dedifferentiated ATSC exhibited more staining in the respective organs compared to control group, and it was thus confirmed that the dedifferentiated ATSC differentiates more.

### <10-3 Confirming Formation of Re-Differentiated Teratoma and In Vivo Differentiation Into Respective Organs

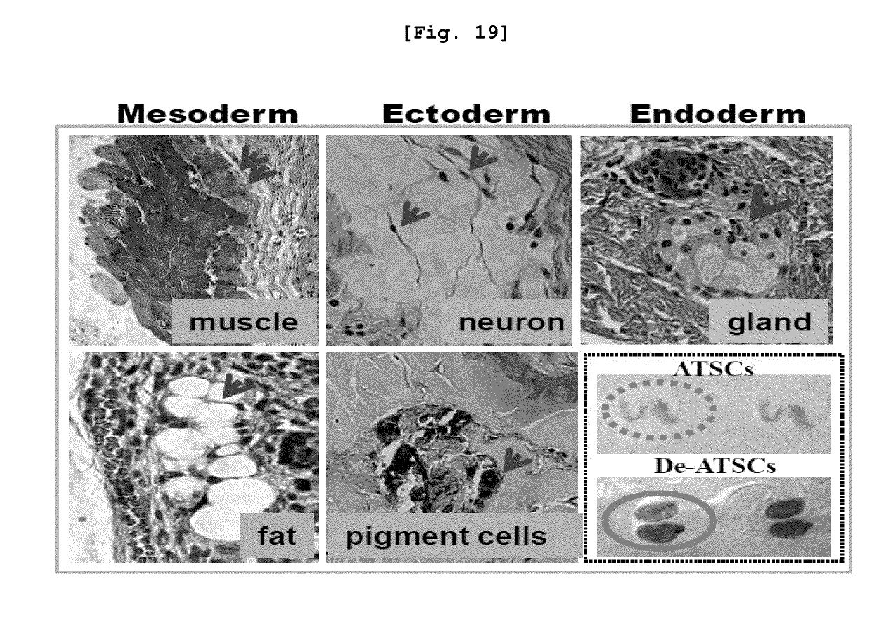

The inventors prepared sections from ATSC or dedifferentiated ATSC-implanted mouse in the same manner as explained above with reference to Example 10-2, and confirmed if teratoma was formed in germline-derived tissues or organs (muscle, nerve, pigment cells, adipocytes and secreting gland). Teratoma is the surest way to prove pluripotency of stem cell. The result of observing the tissue under a microscope is shown in FIG. 19.

As a result, FIG. 19 indicates that teratoma was formed very well in all of the muscle, nerve, pigment cells, adipocytes and secreting glands of the mouse implanted with the dedifferentiated ATSC.

### Example 11

### Confirming Nerve Regenerative Ability of Dedifferentiated ATSC

### <11-1> Confirming In Vitro Nerve Regenerative Ability of Dedifferentiated ATSC

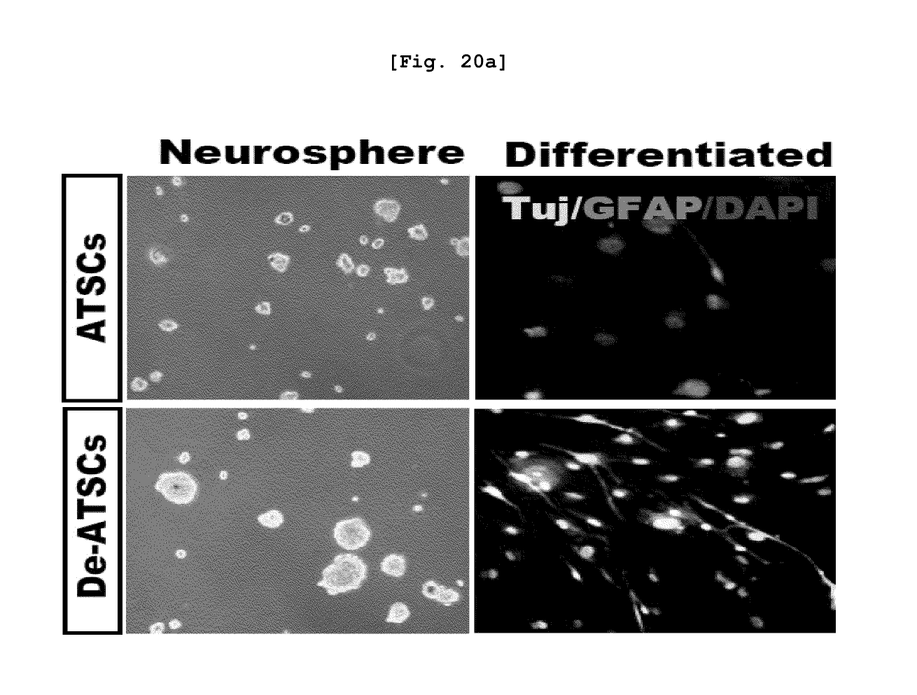

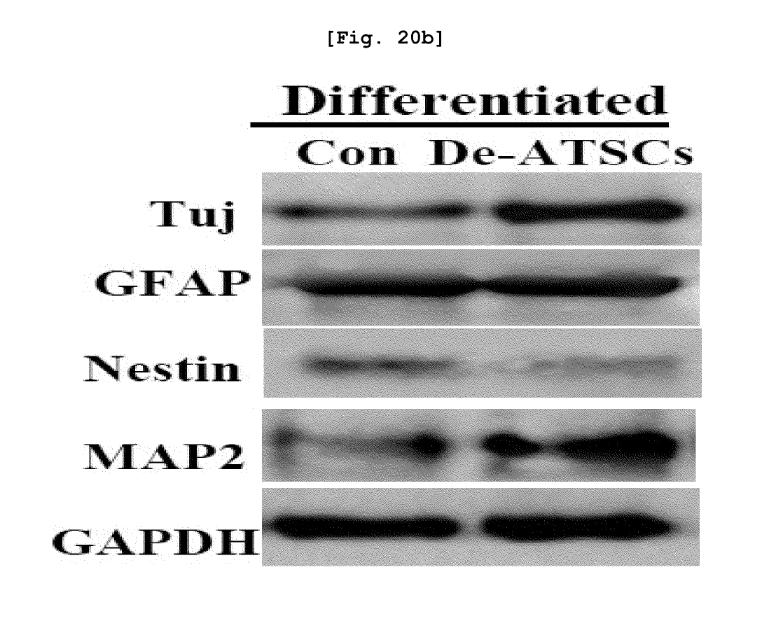

The inventors observed in vitro differentiation of the cells into neurons by immunicytochemical staining and Western blotting to confirm the nerve regenerative ability of dedifferentiated ATSC treated with hypoxia/DHP-derivative. To be specific, experimental cell group and control cell group were differentiated into neurons, as the cells were cultured from 4 to 7 days in a NB culture medium (containing B27, 20 ng/ml bFGF and 10 ng/ml EGF) and thus differentiated into neurosphere (stem cell of central nervous system of brain). The neurosphere was moved onto (PDL-laminin double-coated well plate (Chemicon, U.S.A) and additionally cultured to induce neuron cell differentiation. In order to detect neuron cell marker from the differentiated cells, cells were placed in a fixative of 4% paraformaldehyde, and the fixed cells were reacted in 3% peroxide hydrogen dissolved in 0.1 M PB (pH 7.4) for 30 minutes, and rinsed with PB. The rinsed sections then underwent blocking in a solution containing 1% normal goat serum, 2% BSA, 2% FBS and 0.1% Triton X-100 at room temperature for 30 minutes. Primary antibody (anti TUJ antibody and anti GFAP antibody) was added to the blocking buffer solution and the sample was reacted at 4° C. overnight. Then after rinsing with PB, FITC diluted 1:250, or Texas Red-conjugated secondary antibody was added for reaction. After that, the cells were DAPI stained. The stained results were observed under fluorescent microscope, and the merged result is shown in FIG. 20a.

Additionally, the inventors confirmed expression of Tuj, GFAP, Nestin and MAP2 (microtubule-associated protein 2) genes in the experimental cell group and the control cell group differentiated into neurons by conducting RT-PCR in the same manner as explained above with respect to Example 2-3. GAPDH expression was also observed as a quantitative control group. The primers used in the above-mentioned experiment are shown in Table 9 below. FIG. 20b shows the above results.

TABLE 9

| | Sense primer | | | |
|---|---|---|---|---|
| | | Base sequence | Antisense primer | |
| Gene | SEQ. ID. No. | (5′→3′) | SEQ. ID. No. | Base sequence (5′→3′) |
| Tuj | SEQ. ID. No. 121 | CCTTTGGACACCT | SEQ. ID. No. 122 | GTGAGTGTGTCAG |
| | | ATTCAGG | | CTGGAAG |
| GFAP | SEQ. ID. No. 123 | TCCGCCAAGCCA | SEQ. ID. No. 124 | CATCCCGCATCTC |
| | | AGCACGAAG | | CACAGTCT |
| Nestin | SEQ. ID. No. 125 | AACTGGCACACCT | SEQ. ID. No. 126 | TCAAGGGTATTAC |
| | | CAAGATGT | | CGAAGGGG |
| MAP2 | SEQ. ID. No. 127 | TCAGACTTCCACC | SEQ. ID. No. 128 | AGGGGAAAGATCA |
| | | GAGCAG | | TGGCCC |

As a result, FIG. 20a indicates that dedifferentiated ATSC exhibited more formation of neurosphere and differentiation of neurons. Additionally, as shown in FIG. 20b, dedifferentiated ATSC exhibited increased expression of Tuj, GFAP, and MAP2 compared to the control group, but relatively decreased expression of Nestin.

<11-2> Confirming Nerve Regenerative Ability of Dedifferentiated ATSC in Animal Modes with Nerve Damage

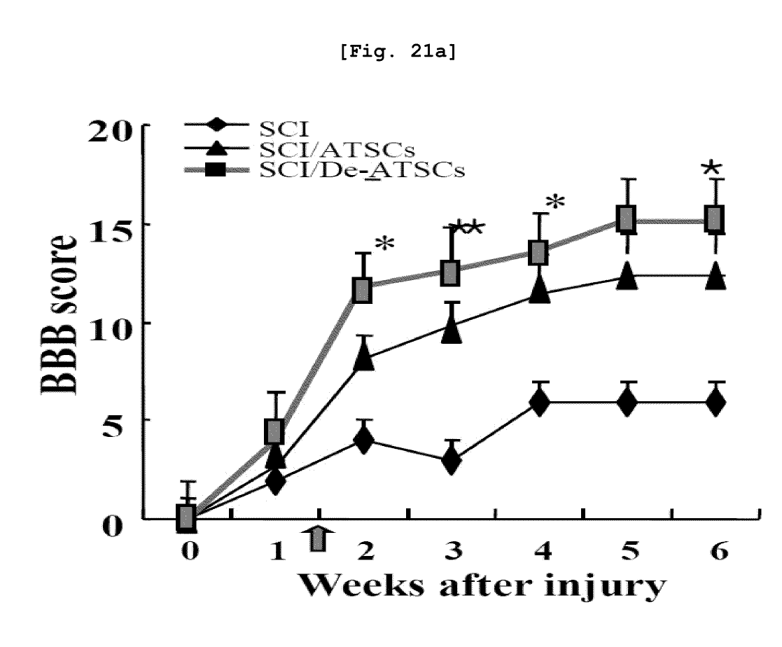

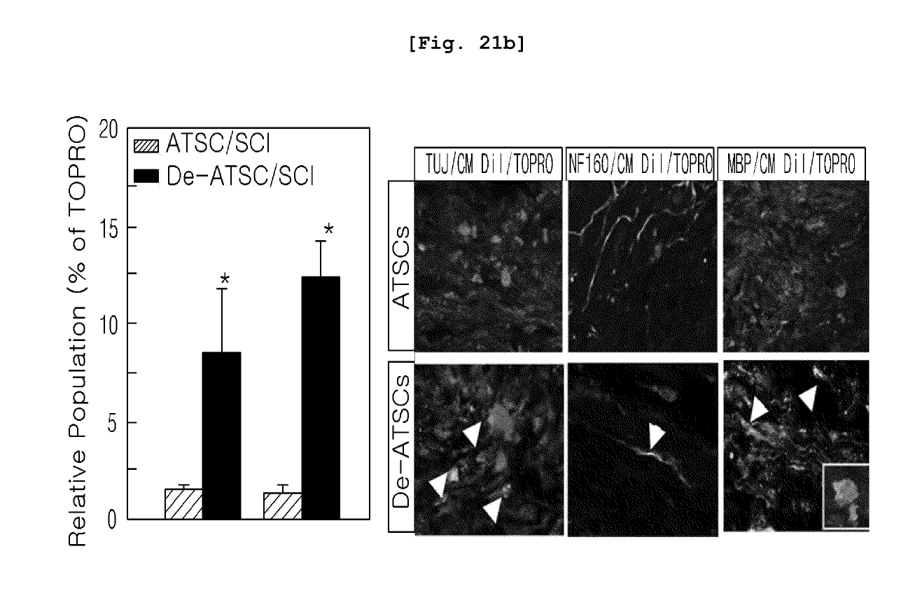

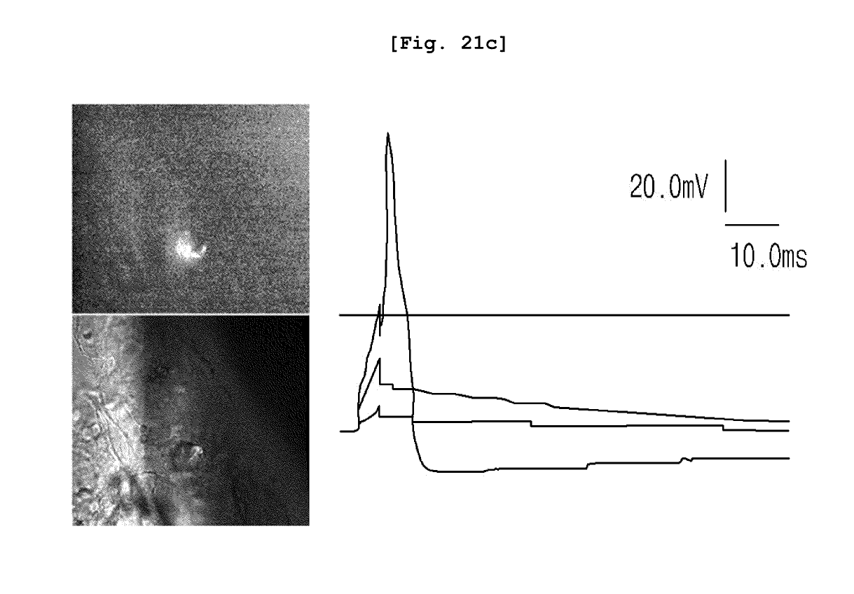

The inventors conducted experiment to confirm if the nerve regenerative ability of dedifferentiated ATSC confirmed in Example 11-1 exhibited the same level of efficacy in the animal models. First, with known method, the inventors prepared SCI (spinal cord injury) rat animal models [Kang S K et al., 2006, Proteomics, 6(9): 2797-2812]. ATSC or dedifferentiated ATSC was implanted into 15 SCI rat models in the same manner as explained above in Example 10-2. Only matrigel was mixed with HBSS and the mixture was implanted into 5 SCI rat models. After implantation, BBB (Basso, Beattie and Bresnahan) scores were measured for 6 weeks in accordance with Table 10 below and the result is expressed in a graphical form as shown in FIG. 21a.

TABLE 10

| Point | Movement details |
|---|---|
| 0 | No movement in hind limb |
| 1 | Slight movement at one or two joints |
| 3 | Noticeable movements observed at two joints (i.e., ankle & knee) |
| 5 | Slight movement observed at two joints and noticeable movement |
| | observed at third joint (i.e., femoral region) |
| 8 | Incapable of supporting the weight of the trunk. Paws dragged, or |
| | paws touch the ground but incapable of supporting the weight of |
| | the trunk |
| 10 | Often capable of supporting weight of the trunk in walking, but |
| | incapable of walking with balanced movements of forelimbs and |
| | hind limbs. |
| 12 | Frequently walking with paws supporting the weight, and |
| | sometimes walk with balanced movements of forelimbs and |
| | hind limbs. |
| 14 | Walking most of times with paws supporting the weight, and |
| | consistently walking with balanced movement of forelimbs and |
| | hind limbs. |
| 15 | Paws sometimes noticeably move off from the ground in walking |
| 16 | Paws noticeably move off from the ground more frequently. |
| 19 | Tail nerve recovered partially. |
| 21 | Tail nerve recovered noticeably. |

The inventors also implanted ATSC and dedifferentiated ATSC into SCI rats prepared in the same manner as explained above with respect to Example 10-2, and observed differentiation of neurons by immunicytochemical staining with the following method. To be specific, 6 weeks after the implantation, 0.9% normal saline solution was refluxed through aorta ascendens of the mouse, and then 0.1 M phosphate buffer (PB, pH 7.4) containing 4% paraformaldehyde was refluxed. After that, organs (i.e., spinal cord) was removed and placed in a fixative at 4° C. The fixed organ was sectioned into 40 μm thickness using microtome. The prepared sections were reacted in 3% peroxide hydrogen dissolved in 0.1 M PB (pH 7.4) for 30 minutes and rinsed with PB. The rinsed sections underwent blocking at room temperature for 30 minutes in a solution containing 1% normal goat serum, 2% BSA, 2% FBS and 0.1% Triton X-100. Primary antibody (anti TUJ antibody, anti NF160 antibody or anti MBP (myelin basic protein) antibody) was added to the blocking buffer solution and reacted at 4° C. overnight. Then after rinsing with PB, FITC diluted 1:250, or Texas Red-conjugated secondary antibody was added for reaction. After that, the sectioned samples were CMDil and TOPRO stained. The stained results were observed under fluorescent microscope, and FIG. 21b shows the respective results in a merged form and the ratio of the CMDil/MBP cells and CMDil/NF160 cells with respect to TOPRO staining.

Additionally, the inventors measured evoked action potential of trans-differentiated neuron prior to sciatic axotomy, immediately after sciatic axotomy and 30 days after sciatic axotomy, respectively, in a SCI rat implanted with ATSC dedifferentiated in the same manner as explained above with respect to Example 10-2. To be specific, under anesthesia, the rat's left sciatic nerve and fourth digital nerve were exposed, stimulating electrode was placed on the proximal sciatic nerve, and electrical activity was induced or recorded by bipolar hooked platinum recording. The evoked action potential in response to the stimulation to ipsilateral sciatic nerve was recorded using Powerlab-800 system (AD Instruments, //www.adinstrumentsinc.com). FIG. 21c shows the recorded results.

As a result, FIG. 21a indicates that the SCI rat implanted with dedifferentiated ATSC had more efficient recovery from nerve injuries than rats implanted with ATSC. Additionally, FIG. 21b indicates that, when hATSC and De-hATSC are compared with each other, De-hATSC exhibited overlapping fluorescent signals with Tuj, NF160 and MBP and thus the treatment effect was confirmed in which De-hATSC cells differentiated from the injured spinal cord into neurons. Additionally, FIG. 21c indicates that the rat implanted with dedifferentiated ATSC into injured spinal cord exhibited action potential in motor nerve by pick, and accordingly, the efficient nerve recovery was confirmed when dedifferentiated ATSC was implanted.

### Example 12

### Confirming Regenerative Ability of Islets of Pancreas by Dedifferentiated ATSC

### <12-1> Preparing Diabetic Mouse Model

To conduct experiment, the inventors used female C57BL6 mice (8˜10 week-old) purchased from Daehan Biolink (Korea). To the terminal veins of the mice feasted for 14 hours or longer, 50 mg of streptozotocin (STZ, Sigma, U.S.A) dissolved in citrate buffer (pH 4.2) per kg was injected and after 14 or 15 days, mice with 10 mM or higher random blood glucose concentration were selected as the mice having developed diabetes and thus used in all the examples explained below. For the selection of the diabetic mice, blood sugar level was measured with Accu-Chda Active blood glucose meter (F. Hoffmann Roche, Swiss) two times a week from the blood samples taken from the tail veins.

### <12-2> Confirming In Vitro Beta Cell Regenerative Ability of Dedifferentiated ATSC

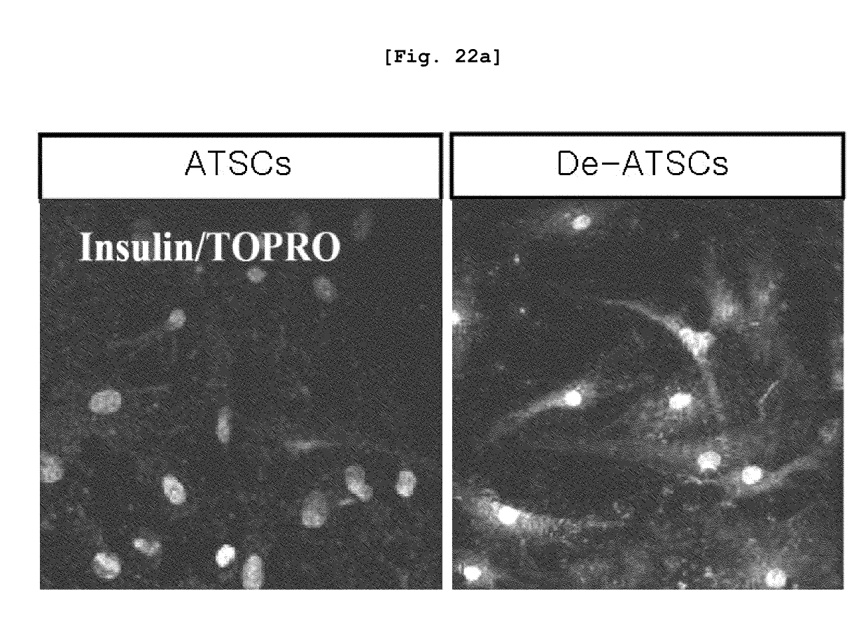

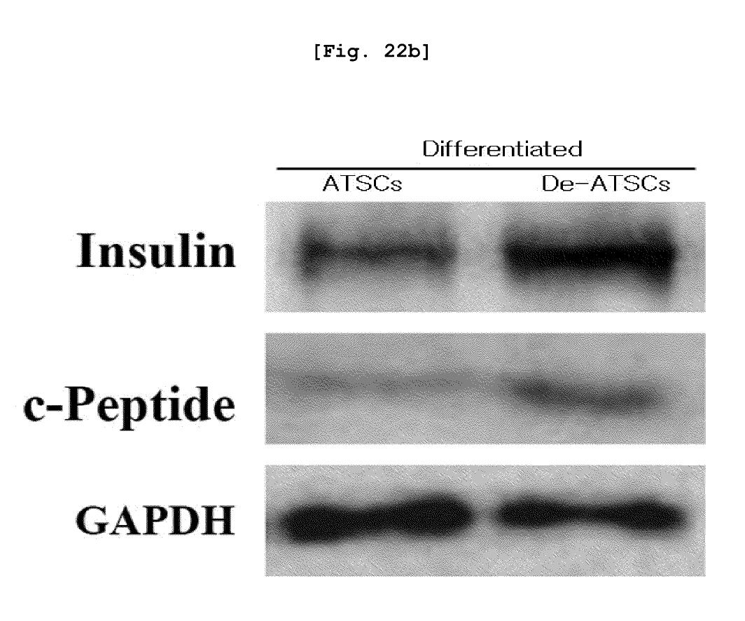

The inventors examined to see if the dedifferentiated ATSC with hypoxia/DHP-derivative treatment are differentiated into beta cells in vitro using immunicytochemical staining and Western blotting, and confirmed the beta cell differentiation ability of the dedifferentiated ATSC. First, in order to differentiate the experimental cell group and control cell group into beta cells, the inventors cultured the cells in a NA+N2 culture medium (containing “N2 media+NA” containing DMEM/F12 supplemented with 10 mM nicotinamide, ITS and B27 media supplement) for 2 weeks so that the cells were differentiated into beta-like cells. The differentiated cells were placed in a fixative of 4% paraformaldehyde, the fixed cells were reacted in 3% peroxide hydrogen dissolved in 0.1 M PB (pH 7.4) for 30 minutes and then rinsed with PB. The rinsed sections underwent blocking at room temperature for 30 minutes in a solution containing 1% normal goat serum, 2% BSA, 2% FBS and 0.1% Triton X-100. The blocked buffer was added with a primary antibody (anti insulin antibody and anti c-peptide antibody) and allowed to react at 4° C. overnight. After rinse with PB, the sample was added with either FITC at dilution of 1:250 or Texas-Red conjugated secondary antibody and allowed to react. The cells were then TOPRO stained. The staining results were observed under a fluorescent microscope. FIG. 22a shows the merged results of such observations.

As a result, FIG. 22a demonstrates that the dedifferentiated ATSC was differentiated into beta cells producing insulin better than control group, and FIG. 22b demonstrates that the beta cells differentiated from the dedifferentiated ATSC exhibited higher insulin and c-peptide expression than control group.

### <12-3> Confirming Pancreatic Islet-Originated Insulin and Beta Cells of the Dedifferentiated ATSC in a Diabetic Mouse

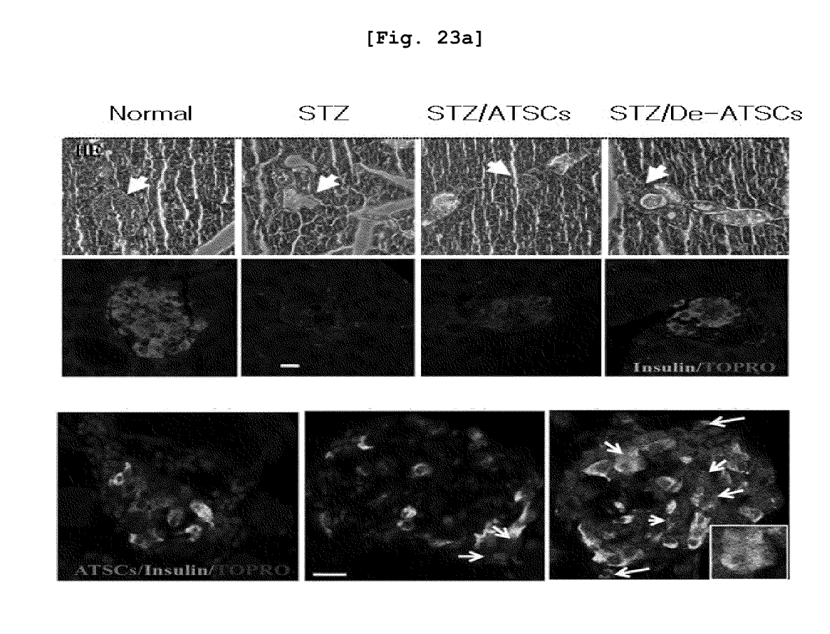

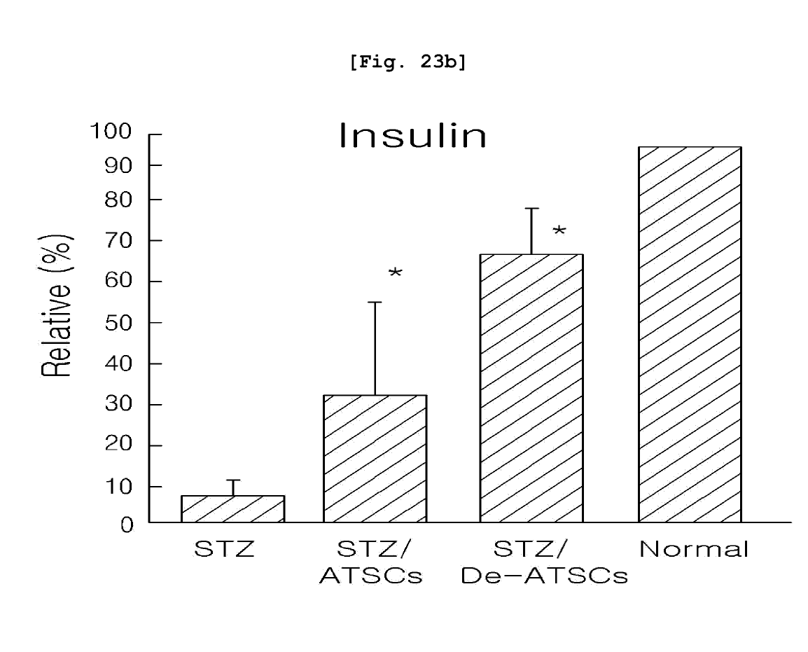

The inventors conducted immunocytochemical staining using anti-insulin antibody respectively for normal mice, diabetic mice prepared in Example 12-1, and diabetic mice implanted with ATSC or dedifferentiated ATSC in the same manner as explained above with reference to Example 11-2, to examine if the pancreatic islet tissue were regenerated. Further, the sectioned tissue samples were H & E and TOPRO stained. Accordingly, the stained results were observed under a fluorescent microscope, and FIG. 23a shows the merged insulin and TOPRO staining results and also H & E staining microscope photographs. Additionally, in order to confirm if insulin is actually secreted by the islet tissue formed in the mouse, the inventors counted the number of insulin positive cells from 20 selected areas of the respective groups. FIG. 23b shows the graphical representation of the ratio of the insulin positive cells of each mouse with respect to the normal mouse.

As a result, the diabetic mouse implanted with dedifferentiated ATSC exhibited more pancreatic islets formed compared to when ATSC was implanted (see FIG. 23a), and more active generation of insulin of the pancreatic islets (see FIG. 23b).

<12-3> Confirming Reduced Blood Sugar Level in Diabetic Mouse Model Injected with Dedifferentiated ATSC

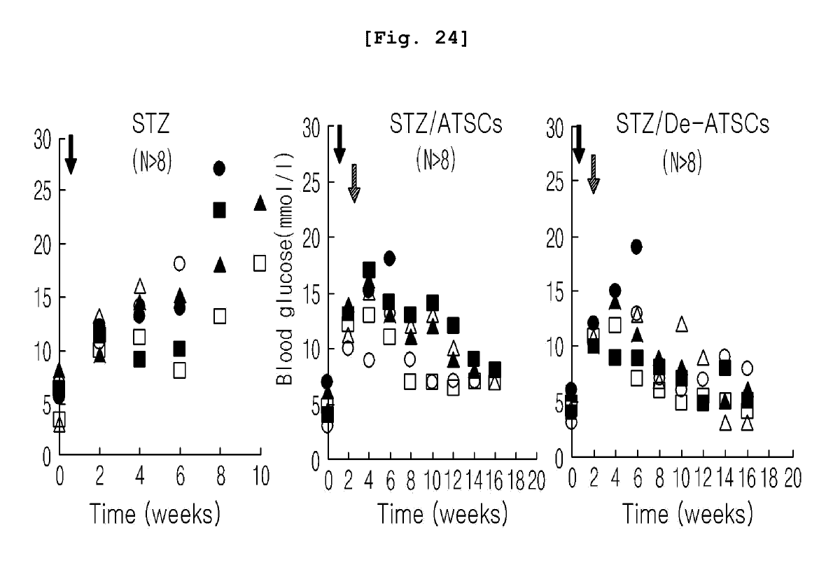

Blood sugar level was measured from blood samples taken from tail veins of at least eight diabetic mice prepared at Example 12-1 and at least 8 mice implanted with ATSC or dedifferentiated ATSC in the same manner as explained above with respect to Example 10-2 at weeks 2, 4, 6, 8, 10, 12, 14, 16, 18 and 20 after the implantation. FIG. 24 shows the graphical representation of the measured results.

As FIG. 24 demonstrates, the diabetic mice implanted with dedifferentiated ATSC exhibited more rapid decrease of blood sugar level.

Accordingly, the inventors could confirm that, with a dedifferentiation method of adipose tissue stromal cells (ATSC) according to an embodiment of the present invention, the ATSC dedifferentiated with the treatment under hypoxia condition and with 4-(3,4-dihydroxy-phenyl)-derivative[4-(3,4-Dihydroxy-phenyl)-derivative] can have pluripotency that enables the ATSC to be differentiated into adipocytes, osteocytes, myocytes, beta cells and cartilage cells. Further, the dedifferentiated cells can be effectively used in the treatment of spinal cord injury and diabetes and in the development of stem cell reaches, tissue regeneration and cytotherapeutic medicines.

What is claimed is:

1. A method for inducing dedifferentiation of cells, the method comprising the steps of:
culturing a differentiated cell to induce differentiation (step 1); and
culturing the cultured cell under hypoxia condition in a culture medium containing 4-(3,4-Dihydroxy-phenyl)-derivative (DHP-derivative) or pharmaceutically-acceptable salt thereof (step 2).

2. The method of claim 1, wherein the DHP-derivative is a compound represented by Chemical formula 1: 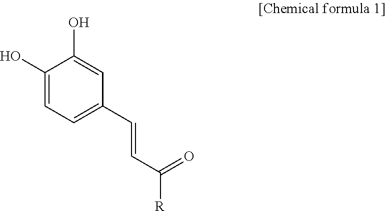
where R is hydrogen or C₁˜C₄ alkyl group.

3. The method of claim 2, wherein the DHP-derivative is a compound represented by Chemical formula 2:

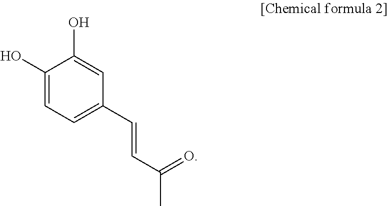

4. The method of claim 1, further comprising the step of culturing the cultured cell of step 2) in a culture condition of dedifferentiated cell.

5. The method of claim 1, wherein the differentiated cell of step 1) is one selected from a group consisting of adipose tissue stromal cells (ATSC), adipose stromal cells (ASC), and differentiated somatic cells.

6. The method of claim of claim 5, wherein the differentiated somatic cells are osteocytes or adipocytes.

7. The method of claim 1, wherein the hypoxia condition comprises a concentration of oxygen ranging from about 0.1% to about 15%.

8. The method of claim 7, wherein the hypoxia condition comprises a concentration of oxygen ranging from about 0.5% to about 5%.

9. The method of claim 1, wherein the hypoxia condition comprises a 1% of oxygen concentration.

10. The method of claim 1, wherein the DHP-derivative or pharmaceutically-acceptable salt thereof of step 2) is contained in a culture medium in an amount of 0.1˜100 ng/ml.

11. The method of claim 1, wherein the DHP-derivative or pharmaceutically-acceptable salt thereof of step 2) is contained in a culture medium in an amount of 1˜50 ng/ml.

12. A pluripotent stem cell dedifferentiated according to the method of claim 1.

13. A cytotherapeutic method for treating an individual comprising the step of administering the pluripotent stem cell of claim 12 into the individual.

14. The cytotherapeutic method of claim 13, used for the treatment of a disease selected from a group consisting of diabetes, connective tissues disease, neuropathic disease, autoimmune disease, muscle damage, osteoporosis and osteoarthritis disease.
